(19) United States
(12) Patent Application Publication
(10) Pub. No.: US 2008/0015224 A1
(43) Pub. Date: Jan. 17, 2008

(54) Nitroxide superoxide dismutase mimetics to treat extracellular superoxide dismutase deficiencies

(76) Inventor: Christopher Wilcox, Great Falls, VA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/823,534

(22) Filed: Jun. 26, 2007

Related U.S. Application Data
(60) Provisional application No. 60/816,655, filed on Jun. 26, 2006.

(51) Int. Cl. A61K 31/445; A61K 31/40; A61P 43/00

(52) U.S. Cl. 514/315; 514/423

(57) ABSTRACT

The invention relates to methods and uses of tempol or other nitroxide superoxide dismutase (SOD) mimetics for the treatment of extracellular-superoxide dismutase (EC-SOD) deficiencies.

### RELATED APPLICATIONS

This application claims the benefit of U.S. Provisional Application No. 60/816,655 filed Jun. 26, 2006, which is hereby expressly incorporated by reference in its entirety.

### STATEMENT REGARDING FEDERALLY SPONSORED R&D

The U.S. Government has a paid-up license in this invention and the right in limited circumstances to require the patent owner to license others on reasonable terms as provided for by the terms of Grant Nos. DK-36079 and DK-49870 awarded by the National Institute of Diabetes and Digestive and Kidney Diseases (NIDDK), and Grant No. HL-68686 awarded by the National Heart, Lung, and Blood Institute (NHLBI).

### FIELD OF THE INVENTION

The invention relates to methods and uses of tempol or other nitroxide superoxide dismutase (SOD) mimetics for the treatment of extracellular-superoxide dismutase (EC-SOD) deficiencies.

### DESCRIPTION OF THE RELATED ART

An increase in reactive oxygen species (ROS) in the blood vessels and kidneys is reported in several experimental animal models of hypertension and in human essential and renovascular hypertension (Wilcox, C S and Ernest H 2005 Am J Physiol Regul Integr Comp Physiol 289:R913-R935). Infusions of angiotensin II (Ang II) increase blood pressure (BP), markers of oxidative stress, and renal expression of the p22^{phox} and Nox-1 components of renal NADPH oxidase (Chabrashvili T et al. 2003 Am J Physiol 285:R117-R124). These effects seem specific for Ang II because similar pressor infusions of norepinephrine into rats do not induce oxidative stress in blood vessels (Laursen J B et al. 1997 Circulation 95:588-593). An increased production of superoxide (O₂.⁻) reduces bioactive NO (Rubanyi G M and Vanhoutte P M 1986 Am J Physiol 250:H822-H827) and contributes to vascular and renal injury in chronic hypertension (Fukai T et al. 2002 Cardiovasc Res 55:239-249; Rajagopalan S et al. 1996 J Clin Invest 97:1916-1923). Superoxide dismutase (SOD) metabolizes O₂.⁻ to H₂O₂ which is further metabolized to inactive products by peroxidases. Hypertension can be moderated or prevented by gene transfer of extracellular (EC)-SOD (Chu Y et al. 2003 Circ Res 92:461-468) or by administration of tempol (Welch W J et al. 2005 Kidney Int 68:179-187) which is a nitroxide SOD mimetic.

### SUMMARY OF THE INVENTION

The invention relates to a method of treating extracellular-superoxide dismutase (EC-SOD) deficiency in humans which comprises administering tempol or other nitroxide superoxide dismutase (SOD) mimetic to a human in need thereof.

A second embodiment of the invention includes methods of treating EC-SOD deficiency in humans comprising identifying a human in need of treatment of EC-SOD deficiency, and administering tempol or other nitroxide SOD mimetic to the human.

A third embodiment of the invention relates to methods of treating EC-SOD deficiency in humans comprising administering tempol or other nitroxide SOD mimetic to a human, and measuring treatment of EC-SOD deficiency in the human.

A fourth embodiment of the invention relates to the use of tempol or other nitroxide SOD mimetic for the preparation of a medicament for treatment of EC-SOD deficiency.

### BRIEF DESCRIPTION OF THE DRAWINGS

FIG. 1. Schematic illustration of the subcellular localization of the three SOD isoforms.

FIG. 2. EC-SOD protein structure. EC-SOD is composed of four domains.

FIG. 3. Chemical formulas of various nitroxide superoxide dismutase mimetics.

FIG. 4: Mean±SEM values for telemetric measurements of MAP in groups of conscious EC-SOD +/+mice (solid circles and continuous lines) and −/−mice (open circles and broken lines) for 6 days before and during subcutaneous infusion of Ang II at a slow pressor rate of 400 ng/kg⁻¹/min⁻¹ (top) or a pressor rate of 1,000 ng/kg⁻/min⁻¹ (bottom) from day 1. Compared with EC-SOD +/+; *P<0.05; **P<0.01.

FIG. 5: Mean±SEM values for glomerular filtration rate, RPF, and RVR in EC-SOD +/+ and −/−mice on day 12 or 13 of infusion of Veh (solid boxes) or Ang II (crosshatch boxes). Compared with Veh in same strain: **P<0.01. Compared with EC-SOD +/+, same treatment group: β indicates P<0.01.

FIG. 6: Mean±SEM values for NOx excretion in EC-SOD+/+ and −/−mice on day 12 or 13 after infusion of Veh (solid boxes) or Ang II (crosshatched boxes). Compared with Veh in same strain: **P<0.01. Compared with EC-SOD +/+, same treatment group: β indicates P<0.01.

FIG. 7: Mean±SEM values for renal excretion of 8-isoprostaglandin F_{2α} and MDA in EC-SOD +/+ and −/−mice on day 12 or 13 of infusion of Veh (solid boxes) or Ang II (crosshatch boxes). Compared with Veh in same strain: ***P<0.005. Compared with EC-SOD +/+, same treatment group: β indicates P<0.01.

FIG. 8: Mean±SEM values for NADPH oxidase activity in of the kidney cortex of EC-SOD +/+ and −/−mice on day 12 or 13 of infusion of Veh (solid boxes) or Ang II (crosshatched boxes) in EC-SOD +/+ and −/−mice. Compared with Veh, in same strain: ***P <0.005. Compared with EC-SOD +/+, same treatment group: β indicates P<0.01.

FIG. 9: Mean±SEM values for protein expression of p22^{phox} (A) or p47^{phox} (B) in the kidney cortex of EC-SOD +/+ and −/−mice on day 12 or 13 of infusion of Veh (solid boxes) or Ang II (cross-hatched boxes). Compared with vehicle in same strain: **P<0.01. Compared with EC-SOD +/+, same treatment group: α indicates p<0.05; β p<0.01.

FIG. 10: Mean±SEM values for SOD activity in plasma, aorta, and kidney cortex of EC-SOD +/+ and −/−mice on day 12 or 13 of infusion of Veh (solid boxes) or Ang II (cross-hatched boxes). Compared with Veh in same strain: **P<0.01. Compared with EC-SOD +/+, same treatment group: a indicates P<0.05. ND, not detected.

FIG. 11: Mean±SEM values for the mRNA (A) or protein (B) expression of EC-SOD in the kidney cortex of EC-SOD +/+mice on day 12 or 13 of infusion of Veh (solid boxes) or Ang II (cross-hatched boxes). Statistics in A from ΔCT method. Compared with Veh: *P<0.05; ***P<0.005.

FIG. 12: Mean±SEM values for the mRNA (A) or protein (B) expression of Mn-SOD or IC-SOD in the kidney cortex of EC-SOD +/+ and −/−mice on day 12 or 13 of infusion of Veh (solid boxes) or Ang II (cross-hatched boxes). Statistics in A from ΔCT method. Compared with EC-SOD +/+, same treatment group: α indicates P<0.05.

FIG. 13: A single nucleotide polymorphism in the gene for extracellular superoxide dismutase predicts an increased cardiovascular death rate. (A) Population frequency and (B) Cardiovascular disease (CVD) risk. Filled bar and filled circle indicate prospectus study of 8,965 normal subjects in Denmark over 10-20 years (after Juul, K. et al. 2004 Circ 109:59-65). Open bar and open circle indicate retrospective study of 456 diabetic hemodialysis patients in Japan (after Yamada, H. et al. 2000 Nephron 84:218-223). Mean±SEM.

FIG. 14: An arginine to glycine substitution at 213 in the EC-SOD gene predicts increased ischemic heart disease events in the Copenhagen heart study and fails to bind to the aorta to reduce the blood pressure (BP) of the spontaneously hypertensive rat (SHR). (A) Copenhagen Heart Study, and (B) Spontaneously Hypertensive Rat (SHR) Study. Referring to panel B, “A” indicates after Juul, K. et al. 2004 Circ 109:59-65 and “B” indicates after Chu, Y. et al. 2005 Circ 112:1047-1053. Mean±SEM.

FIG. 15: Adenoviral transfection into SHR of EC-SOD with an arginine-213 to glycine substitution results in decreased vascular binding and protection from superoxide. (A) Binding to aorta. (B) Aortic superoxide generation. Compared to EC-SOD: *, p<0.05; **, p<0.01. After Cho, Y. et al. 2005 Circ 112:1047-1053. Mean±SEM.

FIG. 16: The extracellular superoxide dismutase knockout mouse has increased blood pressure and increased renal excretion of markers of oxidative stress. (A) MAP, (B) Isprostanes, and (C) MDA. Mean±SEM values (number of mice).

FIG. 17: EC-SOD −/−mice have oxidative stress and increased blood pressure and renal vascular resistance. (A) Isoprostanes, (B) MDA, (C) Conscious MAP, (D) Anesthetized MAP, and (E) RVR. Mean±SEM values (number of mice). Compared to +/+: *, p<0.05; **, p<0.01.

FIG. 18: In vivo EDRF responses of cremasteric vessels are impaired by EC-SOD knockout or prolonged angiotensin II infusion but are restored by superfusion of tempol. Mean±SEM (n=c6 per group). Ang II infusion at 400 ng·kg⁻¹·min⁻¹ sc×12 days. T, 10⁻⁴ M tempol in superfusate. Basal diameter 39±0.4 μm (similar in each group). Compared to EC-SOD +/+; *, p<0.05. Compared to vehicle infusion: †, p<0.05. All effects of tempol, p<0.005.

FIG. 19: Responses of mouse isolated mesenteric resistance vessles to endothelin-1: comparison of EC-SOD +/+ with −/−. (A) Contraction to endothelin-1, (B) Superoxide production with endothelin-1. Mean±SEM values (n=6-8 per group). Response to endothelin-1 in vessels incubated for 20 min with vehicle, tempol (10⁻⁴ M), PEG-SOD (100 units·ml⁻¹), and catalase (300 units·ml⁻¹). Compared to vehicle, ***, p<0.005.

### DETAILED DESCRIPTION OF THE PREFERRED EMBODIMENT

EC-SOD is recognized as a major protection mechanism within the blood vessel wall against vascular damage (Faraci F M and Didion S P 2004 Arterioscler Thromb Vasc Biol 24:1367-1373; Fattman C L et al. 2003 Free Rad Biol Med 35:236-256; Fukai T et al. 2002 Cardiovasc Res 55:239-249). EC-SOD is a secreted protein that metabolizes two molecules of superoxide anion with two protons to form hydrogen peroxide (H₂0₂) and oxygen. It is formed intracellularly and secreted to extracellular sites in blood vessels and organs where it is attached through its heparin binding domain to heparin sulfate proteoglycans located on cell surfaces and the extracellular matrix. It has been discovered that between 2 and 5% of the population have approximately a tenfold increase in plasma EC-SOD levels (Adachi T et al. 1992 Clin Chim Acta 212:89-102; Sandstrom J et al. 1994 J Biol Chem 269:19163-19166; Adachi T et al. 1996 Biochem J 313:235-239; Folz R J et al. 1994 Hum Mol Genet 3:2251-2254). These individuals have an arginine-213 to glycine (R213G) mutation due to a transversion at the first base of codon 213. This alteration reduces the affinity for heparin but does not affect the enzyme activity of EC-SOD. Binding to aortic endothelial cells in culture is reduced fifty-fold by this R213G mutation (Adachi T et al. 1996 Biochem J 313:235-239).

Three clinical association studies have been reported for this mutation. Affected individuals in a Swedish study did not have major phenotypic abnormalities but a trend to increase triglycerides and body weight (Marklund S L et al. 1997 J Intern Med 242:5-14). In Japan, patients with diabetes on hemodialysis carrying this transversion had an increase in 5 year mortality rate with significantly higher death rate from ischemic heart disease and cerebrovascular disease than in non-carriers (Yamada H et al. 2000 Nephron 84:218-223). A recent large study of more than 8000 subjects in Copenhagen detected a 2.3 fold increase in risk of ischemic heart disease in heterozygote carriers of this 213G substitution with a nine-fold increase after adjustment for plasma levels of EC-SOD.

A recent study utilized a transfection approach in the spontaneously hypertensive rat. Whereas transfection of native EC-SOD led to immunostaining in carotid arteries and kidneys, there was minimal staining after transfection with EC-SOD containing the R213G substitution. The primary EC-SOD reduced the blood pressure whereas the R213G substitution did not.

Investigators have found increased plasma levels of EC-SOD in patients on hemodialysis compared to age match controls. This increase is associated with oxidative stress and endothelial dysfunction as indicated by high levels of circulating asymmetric dimethyl arginine. This may represent one of several acquired defects of EC-SOD binding to the blood vessel wall. Indeed, studies have indicated that homocysteine, which is elevated in cardiovascular disease and especially in patients on hemodialysis, impairs the binding of EC-SOD to endothelial cells (Yamamoto M et al. 2000 FEBS Lett 486:159-162). In addition, nitric oxide and its decomposition derivatives also decreased the EC-SOD binding to endothelial cells (Yamamoto M et al. 2001 FEBS Lett 505:296-300).

Collectively, these data suggest that a failure of binding of EC-SOD to blood vessels increases plasma levels of EC-SOD. This can be detected by an increased plasma levels. It may be perhaps the most potent genetic abnormality predisposing individuals to cardiovascular disease. Its frequency of 2-5% in the population indicates that it is remarkably prevalent. Other evolving data suggests that, in addition to these genetic defects, acquired defects in EC-SOD binding may occur in the context of oxidative stress, hyperhomocysteinemia, chronic renal failure, and alterations in blood vessel NO bioavailability and oxidation. Currently there is no therapy directed towards correcting this defect.

The present invention provides the use of nitroxide SOD mimetics (e.g., tempol) to treat EC-SOD deficiency in patients with genetic or acquired defects in EC-SOD, as indicated by high plasma levels of EC-SOD and/or the genetic polymorphism.

The expression of EC-SOD in blood vessels is reduced by NO deficiency (Fukai T et al. 2000 J Clin Invest 105:1631-1639), transforming growth factor β (Marklund S L 1992 J Biol Chem 267:6696-6701), hyperhomocysteinemia (Nonaka H et al. 2001 Circ 104:1165-1170) and in patients with coronary artery disease (Landmesser U et al. 2000 Circ 101:2264-2270). Some 2% to 5% of the normal population (Chu Y et al. 2005 Circ 112:1047-1053) and 13% of diabetics with end stage renal disease (Yamada H et al. 2000 Nephron 84:218-223) carry a substitution of arginine-213 by glycine,. which prevents the binding of EC-SOD to blood vessels and thereby enhances endothelial NO bioinactivation by O₂.⁻ (Chu Y et al. 2005 Circ 112:1047-1053). Normal subjects or diabetic patients with end stage renal disease who express this arginine-213 by glycine substitution have almost a doubled risk of ischemic heart disease (Yamada H et al. 2000 Nephron 84:218-223; Juul K et al. 2004 Circ 109:59-65). The EC-SOD −/−mouse is a good model for human subjects with ineffective EC-SOD expression. Our finding that this EC-SOD −/−mouse has an elevated BP, renal vasoconstriction, oxidative stress, increased p22^{phox} expression and NADPH oxidase activity in the kidney confirms that EC-SOD can be an important factor underlying cardiovascular disease. Moreover, it suggests a therapeutic role for EC-SOD (Hatori N et al. 1992 Free Radic Biol Med 13:221-230) or for SOD mimetics such as the nitroxide, tempol (Welch W J et al. 2005 Kidney Int 68:179-187) to restore SOD activity in those conditions associated with reduced EC-SOD expression or activity.

Definitions

Unless defined otherwise, technical and scientific terms used herein have the same meaning as commonly understood by one of ordinary skill in the art to which this invention belongs. See, e.g., Dorland's illustrated medical dictionary (30^{th} Edition), D. M. Anderson, P. D. Novak, J. Keith and M. A. Elliott, Eds. Saunders (an Imprint of Elsevier), Philadelphia, Pa., 2003.

Superoxide Dismutases: Basic Characteristics and Functions

In mammals, there are three isoforms of SOD, and each are products of distinct genes but catalyze the same reaction:
2H⁺+2O₂⁻.→H₂O₂+O₂

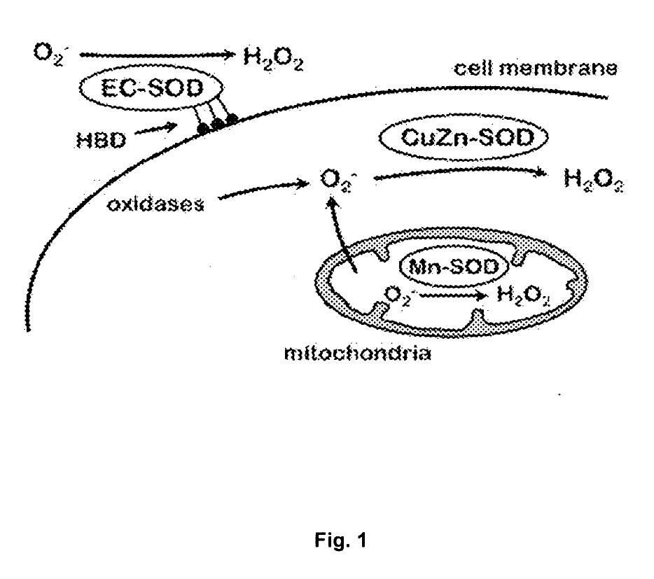

The three isoforms of SOD are cytosolic or copper-zinc SOD (CuZn-SOD or SOD-1), manganese SOD (Mn-SOD or SOD-2) localized in mitochondria, and an extracellular form of CuZn-SOD (EC-SOD or SOD-3) (FIG. 1). Although the subcellular localization of each isoform of SOD is unique, only very recently have studies begun to focus on the functional importance of individual SOD isoforms within the vessel wall under normal conditions or during vascular disease. Expression and activity of SODs presumably have a profound effect on responses of vascular cells to both acute and chronic oxidative stress. Compartmentalization of various reactive oxygen species (ROS) may be very important in relation to overall effects. Recent studies in nonvascular cells suggest the different isoforms of SOD have major but distinctive roles.

As indicated above, SODs dismute superoxide into hydrogen peroxide plus molecular oxygen. There are several functional consequences of this enzymatic activity. First, SODs protect against superoxide-mediated cytotoxicity, such as inactivation of mitochondrial proteins containing iron-sulfur (Fe—S) centers (e.g., aconitase and fumarase). Such interactions are of potential importance, as damage to such complexes results in release of free iron and subsequent formation for hydroxyl radical (a highly reactive ROS).

NO reacts with superoxide at a rate 3 times faster than dismutation of superoxide by SOD. Because of the efficiency of the reaction (superoxide reacts with NO more efficietly than with any other known molecule), the local concentration of SOD is a key determinant of bioactivity (the biological half-life) of NO. Thus, a second major function of SOD is to protect NO and NO-mediated signaling. Multiple lines of evidence have shown that NO signaling plays a major role in vascular biology. In addition to inactivating NO and thus preventing NO-mediated signaling, the reaction of NO with superoxide produces peroxynitrite, a potent oxidant with the potential to produce cytotoxicity. Emerging evidence suggests that formation of peroxynitrite has multiple effects, including (1) selective nitration of tyrosine residues in proteins, such as prostacyclin synthase and Mn-SOD, (2) activation of poly (ADP-ribose) polymerase (PARP) and expression of inducible NO synthase (iNOS), potentially important mediators of vascular dysfunction in disease states, (3) oxidation of tetrahydrobiopterin, and (4) oxidation of the zinc-thiolate complex in endothelial NOS (eNOS). The latter two effects can produce eNOS “uncoupling,” a condition in which the normal flow of electrons within the enzyme is diverted such that eNOS produces superoxide rather than NO.

A third functional consequence of SOD activity is formation of hydrogen peroxide. The importance of this ROS within vascular cells is becoming increasingly apparent. Hydrogen peroxide is relatively stable and diffusible (including through cell membranes), compared with many other ROS. These features make hydrogen peroxide somewhat analogous to NO as a signaling molecule. For example, hydrogen peroxide is a signaling molecule and regulator of gene expression and may be an important mediator of hypertrophy of vascular muscle in response to stimuli such as angiotensin II. Hydrogen peroxide can activate select transcription factors and may also function as an endothelium-derived hyperpolarizing factor (EDHF) in some blood vessels, but has also been suggested to be an endothelium-derived relaxing factor (EDRF) without functioning as an EDHF. In combination with some transition metals like iron or copper, hydrogen peroxide can react to form hydroxyl radical, a highly reactive ROS, and thus produce cellular injury via the Fenton reaction. Hydrogen peroxide mediated effects and local concentrations are regulated by activity of the various glutathione peroxidases or catalase.

Thus, SOD plays an important role in vascular biology. Because compartmentalization of superoxide presumably is of fundamental importance in relation to overall effects, the functional importance of individual SOD isoforms has begun to be studied.

EC-SOD (SOD-3)

EC-SOD is the only isoform of SOD that is expressed extracellularly, binding to tissues via its heparin-binding domain that provides affinity of the protein for heparan sulfate proteoglycans on cell surfaces, in basal membranes, and in the extracellular matrix. EC-SOD is localized throughout the vessel wall, particularly between endothelium and vascular muscle. The major source of the protein is thought to be vascular muscle. Endothelium does not appear to produce EC-SOD.

Unlike some tissue, such as brain, EC-SOD accounts for a large portion of total SOD activity in blood vessels. Expression of EC-SOD in vascular cells and within the vessel wall can be altered in response to a variety of stimuli including exercise, growth factors, cytokines, vasoactive stimuli including angiotensin II and NO, and homocysteine as well as during hypertension, atherosclerosis, and diabetes. In contrast to CuZn-SOD, expression of EC-SOD has been reported to increase in regions of the vasculature with disturbed blood flow. In addition to changes in production or excretion of EC-SOD or both, the binding of EC-SOD to tissue can be altered by factors such as NO and homocysteine as well as genetic factors such as polymorphisms in the heparin binding domain (HBD).

Measurements of EC-SOD release into plasma in response to heparin are commonly used as an index of vascular bound EC-SOD. It should be noted that the amount of EC-SOD that is released by heparin using this approach is thought to be only a small fraction of total vascular EC-SOD. Although this approach, along with measurements of EC-SOD expression or activity, has been used in numerous studies, there is still relatively little known about the functional importance of this SOD isoform. Based on its extracellular localization, it has been hypothesized that at least 1 major function of EC-SOD is to protect NO as it diffuses from endothelium to its major target (soluble guanylate cyclase) in vascular muscle. Although defined as being extracellular, some evidence suggests that EC-SOD may also be expressed intracellularly.

Diethyldithiocarbamate (DDC) has been used commonly in studies of vascular biology, but DDC does not distinguish between EC-SOD and CuZn-SOD in its effects. Thus, other approaches are needed to define the role of EC-SOD. To date, these approaches have consisted mainly of studies using genetically-altered mice and overexpression of EC-SOD using viral-mediated gene transfer. In aorta of EC-SOD-deficient mice, there was increased superoxide, impaired basal activity of NO, and impaired endothelium-dependent relaxation. The response to an endothelium-dependent agonist (acetylcholine) is that these mice are not altered in small pulmonary arteries and only very modestly attenuated in the cerebral microcirculation. Deficiency in EC-SOD does not alter baseline blood pressure but increases arterial pressure in two models of hypertension that are greater in EC-SOD-deficient mice than in controls. Vasoconstrictor responses to serotonin are augmented in EC-SOD-deficient mice.

Studies using overexpression strategies have revealed protective effects of EC-SOD on blood vessels. Gene transfer of EC-SOD reduced vascular superoxide levels during atherosclerosis in spontaneously hypertensive rats (SHR) and in an lipopolysaccharide (LPS)-induced model of inflammation. Effects of overexpression of EC-SOD using this approach on endothelial function have varied. For example, gene transfer of EC-SOD increased basal NO bioactivity in stroke-prone SHR, enhanced endothelium-dependent relaxation in SHR and after LPS, but did not improve endothelial function in atherosclerosis. The presence of the heparin-binding domain was necessary for EC-SOD to exert protective effects. Overexpression of EC-SOD with this viral approach or using transgenic mice attenuates vasospasm after subarachnoid hemorrhage.

It has been suggested that EC-SOD is the major determinant of NO bioavailability in blood vessels. In this regard, it is noteworthy that effects of deficiency in EC-SOD and CuZn-SOD on endothelial function are similar. Thus, to protect NO over its entire diffusion route (from site of production within endothelium to its major molecular target in vascular muscle), normal expression of both CuZn-SOD and EC-SOD may be essential.

In relation to EC-SOD, it is important to recall that most previous studies of vascular oxidative stress have been performed in the rat, and there are species differences in relation to vascular EC-SOD content. For example, whereas blood vessels from mice and humans have similar levels of EC-SOD, the rat has very low vascular levels of EC-SOD compared with most other mammalian species that have been studied. This phenotype in the rat is caused by a difference in a key amino acid affecting protein subunit interaction. In contrast to other species, rat EC-SOD has a low affinity for heparin and does not bind to heparan sulfate under physiological conditions. Thus, the rat essentially lacks vascular EC-SOD. Studies of blood vessels in the rat therefore have a potential limitation in that the species may not be particularly representative of other species, including humans, that express normal levels of EC-SOD in the vasculature. Conversely, this phenotype in the rat may be advantageous for experimental studies, as these animals may exhibit increased susceptibility to oxidative stress (i.e., greater endothelial dysfunction, larger increases in arterial pressure, etc) than other commonly studied animal models (or humans). In this context, it may seem surprising that rats are very resistant to development of atherosclerosis, as deficiency in EC-SOD might be expected to promote atherosclerosis. However, recent work in gene targeted mice suggests that EC-SOD deficiency has no effect on development of atherosclerosis. Thus, other differences in gene expression or genetic background are more likely to account for resistance to atherosclerosis in rats.

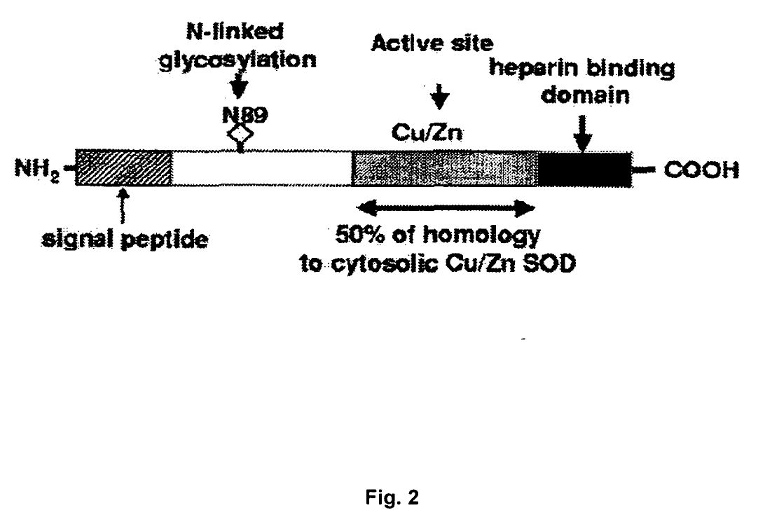

In most species, EC-SOD is a tetramer composed of two disulfide-linked dimers. Each subunit has a molecular weight of ˜30 KDa and is composed of an amino-terminal signal peptide which permits secretion from the cell, an active domain which binds copper and zinc, and a carboxy-terminal region which is involved in binding to sulfated proteoglycan (FIG. 2). Firstly, an amino-terminal signal peptide permits secretion from the cell. Secondly, an N-linked glocosylation site at Asn-89 is useful in the separation of EC-SOD from cytosolic Cu/Zn SOD and greatly increases the solubility of the protein. Thirdly, the domain containing active site (amino acid residues 96-193) shows about 50% homology to Cu/Zn SOD. All the ligands to Cu and Zn and the arginine in the entrance to the active site in Cu/Zn SOD can be identified in this domain of EC-SOD. Finally, a C-terminal region corresponding to the heparin-binding domain has a cluster of positively charged residues. This region is critical for binding to heparin sulfate glycosaminoglycans. The nucleotide and amino acid sequences of human EC-SOD are given by Genbank accession number J02947.

EC-SOD Genetic Polymorphisms

Extracellular-superoxide dismutase (EC-SOD) is a secretory, tetrameric copper- and zinc-containing glycoprotein with a subunit molecular mass of about 30 kDa. EC-SOD is the major SOD isoenzyme in plasma, lymph and synovial fluid, but occurs primarily in tissues, anchored to heparan sulphate proteoglycans in the glycocalyx of cell surfaces and in the connective tissue matrix; this form of the enzyme accounts for over 90% of the EC-SOD. EC-SOD in plasma is heterogeneous with regard to heparin affinity and can be divided into five fractions: form I, which lacks affinity; forms II and III, with weak affinity; and forms IV and V, with relatively strong affinity in heparin-HPLC. Data have indicated that EC-SOD form V is the primary form synthesized in the body and that EC-SOD forms I-IV, with reduced heparin affinity seen in plasma, are the result of endo- and exo-proteolytic truncations at the C-terminal end. The C-terminal portion of EC-SOD, which contains three lysines, six arginines and a histidine in the last 21 amino acids, is responsible for the heparin affinity of the enzyme. In particular, the cluster of six basic amino acids, Arg-210-Arg-215, forms an essential part of the heparin-binding domain.

Ninety nine percent of EC-SOD is anchored to heparan sulfate proteoglycans in the tissue interstitium, and 1% is located in the vasculature in equilibrium between the plasma and the endothelium. In a Swedish study, analysis of EC-SOD in plasma samples from 504 random blood donors revealed a common (2.2%) phenotypic variant displaying 10-fold increased plasma EC-SOD content (Sandstrom J et al. 1994 J Biol Chem 269:19163-19166). In the Swedish study, serum EC-SOD levels from healthy persons were clearly divided into two groups: a low-concentration group below about 200 ng/ml and a high-concentration group above about 200 ng/ml. The low concentration group showed serum EC-SOD levels that averaged around 150, although some members within the group distribution had values of 200 ng/ml, 250 ng/ml and 300 ng/ml. The EC-SOD in the plasma of these individuals, collected both before and after intravenous injection of heparin, displayed a reduced heparin affinity when compared with samples from normal individuals. The specific enzymatic activity was the same as that of normal enzyme. Nucleotide sequence analyses of two of the affected subjects revealed a nucleotide exchange consisting of a single-base substitution C→G at position 760 of the cDNA of human EC-SOD resulting in a substitution of Arg-213 by Gly (R213G). The substitution is located in the center of the carboxyl-terminal cluster of positively charged amino acid residues, which defines the heparin-binding domain. Polymerase chain reaction-single-strand conformational polymorphism and allele-specific polymerase chain reaction showed that all 11 affected individuals were heterozygous, carrying the same single-base mutation. Recombinant EC-SOD containing this mutation had a reduced heparin affinity similar to that of EC-SOD from variant persons. The high plasma activity can be explained by an accelerated release from the tissue interstitium heparan sulfate to the vasculature and should thus be accompanied by significantly reduced tissue EC-SOD activities.

Serum EC-SOD levels from healthy persons are clearly divided into two groups: a low-concentration group (Group I, below 400 ng/ml) and a high-concentration group (Group II, above 400 ng/ml) (Adachi T. et al. 1996 Biochem J 313:235-240). A family study in Japan corroborated the genetic transmission of a high EC-SOD level in serum. Investigators found that ˜6.4% of a population tested were high plasma-level EC-SOD donors (Group II). In patients with various diseases, EC-SOD seemed to be divided into the above two groups. The frequency of Group II was significantly greater for haemodialysis patients than for healthy persons or other patients. In Japanese individuals having high serum EC-SOD, the same R213G gene mutation was also found. The 45 donors with a high serum level of EC-SOD were heterozygotes for the R213G mutation. Three homozygotes were found in haemodialysis patients.

Measuring EC-SOD Genetic Polymorphisms

In an embodiment of the present invention there is a method of detecting susceptibility to development of diseases related to EC-SOD deficiency in an individual, comprising the steps of obtaining a sample from the individual and assaying the nucleic acid sequence for an R213G mutation, wherein the presence of the mutation in the nucleic acid sequence indicates that the individual is susceptible to development of diseases related to EC-SOD deficiency.

A skilled artisan recognizes that there are a variety of methods to detect a mutation in a nucleic acid sequence. Methods regarding allele-specific probes for analyzing particular nucleotide sequences are described in the literature. Allele-specific probes are typically used in pairs. One member of the pair shows perfect complementarity to a wildtype allele and the other members to a variant allele. In idealized hybridization conditions to a homozygous target, such a pair shows an essentially binary response. That is, one member of the pair hybridizes and the other does not. An allele-specific primer hybridizes to a site on target DNA overlapping the particular site in question and primes amplification of an allelic form to which the primer exhibits perfect complementarity. This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarity to a distal site. The single-base mismatch impairs amplification and little, if any, amplification product is generated.

Particular nucleic acid sites can also be identified by hybridization to oligonucleotide arrays. An example described in the literature includes arrays having four probe sets. A first probe set includes overlapping probes spanning a region of interest in a reference sequence. Each probe in the first probe set has an interrogation position that corresponds to a nucleotide in the reference sequence. That is, the interrogation position is aligned with the corresponding nucleotide in the reference sequence when the probe and reference sequence are aligned to maximize complementarity between the two. For each probe in the first set, there are three corresponding probes from three additional probe sets. Thus, there are four probes corresponding to each nucleotide in the reference sequence. The probes from the three additional probe sets are identical to the corresponding probe from the first probe set except at the interrogation position, which occurs in the same position in each of the four corresponding probes from the four probe sets, and is occupied by a different nucleotide in the four probe sets. Such an array is hybridized to a labeled target sequence, which may be the same as the reference sequence, or a variant thereof. The identity of any nucleotide of interest in the target sequence can be determined by comparing the hybridization intensities of the four probes having interrogation positions aligned with that nucleotide. The nucleotide in the target sequence is the complement of the nucleotide occupying the interrogation position of the probe with the highest hybridization intensity.

The literature also describes subarrays that are optimized for detection of variant forms of a precharacterized nucleotide site. A subarray contains probes designed to be complementary to a second reference sequence, which can be an allelic variant of the first reference sequence. The second group of probes is designed by the same principles as above except that the probes exhibit complementarity to the second reference sequence. The inclusion of a second group can be particularly useful for analyzing short subsequences of the primary reference sequence in which multiple mutations are expected to occur within a short distance commensurate with the length of the probes (i.e., two or more mutations within 9 to 21 bases).

An additional strategy for detecting a particular nucleotide site uses an array of probes as described in the literature. In this strategy, an array contains overlapping probes spanning a region of interest in a reference sequence. The array is hybridized to a labeled target sequence, which may be the same as the reference sequence or a variant thereof. If the target sequence is a variant of the reference sequence, probes overlapping the site of variation show reduced hybridization intensity relative to other probes in the array. In arrays in which the probes are arranged in an ordered fashion stepping through the reference sequence (e.g., each successive probe has one fewer 5′ base and one more 3′ base than its predecessor), the loss of hybridization intensity is manifested as a “footprint” of probes approximately centered about the point of variation between the target sequence and reference sequence.

A method for determining the identity of the nucleotide present at a particular site that employs a specialized exonuclease-resistant nucleotide derivative is described in the literature. A primer complementary to the allelic sequence immediately 3′ to the site is permitted to hybridize to a target molecule obtained from a particular animal or human. If the site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the site of the target molecule was complementary to that of the nucleotide derivative used in the reaction. The Mundy method has the advantage that it does not require the determination of large amounts of extraneous sequence data. It has the disadvantages of destroying the amplified target sequences, and unmodified primer and of being extremely sensitive to the rate of polymerase incorporation of the specific exonuclease-resistant nucleotide being used.

A solution-based method for determining the identity of the nucleotide of a particular site is described by the literature. A primer is employed that is complementary to allelic sequences immediately 3′ to the site. The method determines the identity of the nucleotide of that site using labeled dideoxynucleotide derivatives, which, if complementary to the nucleotide of the site will become incorporated onto the terminus of the primer.

An alternative method, known as Genetic Bit Analysis or “GBA” is also described by the literature. The method uses mixtures of labeled terminators and a primer that is complementary to the sequence 3′ to a site in question. The labeled terminator that is incorporated is thus determined by, and complementary to, the nucleotide present in the site of the target molecule being evaluated. The method is preferably a heterogeneous phase assay, in which the primer or the target molecule is immobilized to a solid phase. It is thus easier to perform, and more accurate.

An alternative approach, the “Oligonucleotide Ligation Assay” (“OLA”) has also been described as capable of detecting a nucleotide sequence variation. The OLA protocol uses two oligonucleotides which are designed to be capable of hybridizing to abutting sequences of a single strand of a target. One of the oligonucleotides is biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. A nucleic acid detection assay that combines attributes of polymerase chain reaction (PCR) and OLA is also described by the literature. In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Recently, several primer-guided nucleotide incorporation procedures for assaying particular sites in DNA have been described in the literature.

In an additional specific embodiment of the present invention an assaying step is by antibody detection with antibodies to the R213G mutation of the EC-SOD protein. In a particular embodiment of the present invention an assaying step is by monoclonal antibody detection with monoclonal antibodies to the R213G mutation of the EC-SOD protein.

Mismatch Oligonucleotide Mutation Detection

A skilled artisan recognizes that one method to identify a point mutation in a nucleic acid sequence is by mismatch oligonucleotide mutation detection, also referred to by other names such as oligonucleotide mismatch detection. In a specific embodiment, a nucleic acid sequence comprising the site to be assayed for the mutation is amplified from a sample, such as by polymerase chain reaction, and a mutation is detected with mutation-specific oligonucleotide probe hybridization of Southern or slot blots, or a combination thereof.

In a specific embodiment of the present invention, an R213G mutation in EC-SOD nucleic acid sequence is identified by methods and/or kits employing oligonucleotide mismatch detection.

Single-Strand Conformation Polymorphism

Single-strand conformation polymorphism (SSCP) facilitates detection of polymorphisms, such as single base pair transitions, through mobility shift analysis on a neutral polyacrylamide gel by methods well known in the art. In specific embodiments, the method is subsequent to polymerase chain reaction or restriction enzyme digestion, either of which is followed by denaturation for separation of the strands. The single stranded species are transferred onto a support such as a nylon membrane, and the mobility shift is detected by hybridization with a nick-translated DNA fragment or with RNA. In alternative embodiments, the single stranded product is itself labeled, such as with radioactivity, for identification. Samples manifesting migration shifts in SSCP gels in a specific embodiment are analyzed further by other well known methods, such as by DNA sequencing.

In a specific embodiment of the present invention, an R213G mutation in EC-SOD nucleic acid sequence is identified by methods and/or kits employing single-strand conformation polymorphism.

Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in the literature. Other methods of hybridization that may be used in the practice of the present invention are disclosed in the literature.

Amplification of Nucleic Acids

Nucleic acids used as a template for amplification may be isolated from cells, tissues or other samples according to standard methodologies (Sambrook et al. Molecular Cloning: A Laboratory Manual, Cold Spring Harbor Laboratory, Cold Spring Harbor, N.Y., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to EC-SOD wildtype or mutant are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR) which is described in detail in the literature.

A reverse transcriptase PCR amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in the literature. Polymerase chain reaction methodologies are well known in the art. Representative methods of RT-PCR are also described in the literature.

Another method for amplification is ligase chain reaction (“LCR”), disclosed in the literature. Also described by the literature is a method similar to LCR for binding probe pairs to a target sequence. A method based on PCR and oligonucleotide ligase assay (OLA) disclosed in the literature may also be used.

Qbeta Replicase, described in the literature, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention. Strand Displacement Amplification (SDA), disclosed in the literature, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR. Also disclosed in the literature is a nucleic acid amplification process involving cyclically synthesizing single-stranded RNA (“ssRNA”), ssDNA, and double-stranded DNA (dsDNA), which may be used in accordance with the present invention.

Also disclosed in the literature is a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR”.

Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al., 1989. One example of the foregoing described in the literature discloses an apparatus and method for the automated electrophoresis and transfer of nucleic acids. The apparatus permits electrophoresis and blotting without external manipulation of the gel and is ideally suited to carrying out methods according to the present invention.

Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

An RNase A mismatch cleavage assay described in the literature involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in the literature.

Kits

All the essential materials and/or reagents required for detecting EC-SOD wildtype or mutant sequences in a sample may be assembled together in a kit. This generally will comprise a probe or primers designed to hybridize specifically to individual nucleic acids of interest in the practice of the present invention, including EC-SOD wildtype or mutant sequences. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

Immunodetection Methods

Immunobinding methods include methods for detecting and quantifying the amount of a wild-type or mutant EC-SOD protein reactive component in a sample and the detection and quantification of any immune complexes formed during the binding process. Here, one would obtain a sample suspected of containing a wild-type or mutant EC-SOD protein and/or peptide, and contact the sample with an antibody against wild-type or mutant EC-SOD, and then detect and quantify the amount of immune complexes formed under the specific conditions.

In terms of antigen detection, the biological sample analyzed may be any sample that is suspected of containing a wild-type or mutant EC-SOD protein-specific antigen, such as any tissue or specimen, a homogenized tissue extract, a cell, separated and/or purified forms of the wild-type or mutant EC-SOD protein-containing sample(s), or even any biological fluid that comes into contact with the tissue.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any EC-SOD protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The EC-SOD antibody employed in the detection may itself be linked to a detectable label, wherein one would then simply detect this label, thereby allowing the amount of the primary immune complexes in the sample to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the method described in the previous paragraph up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the detection of susceptibility to development of diseases related to EC-SOD deficiency in an individual. Here, a biological and/or clinical sample suspected of containing a wild-type or mutant EC-SOD protein, polypeptide, peptide and/or mutant is used.

In the detection of susceptibility to development of diseases related to EC-SOD deficiency in an individual, the detection of EC-SOD mutant, and/or an alteration in the levels of EC-SOD, in comparison to the levels in a corresponding biological sample from a normal subject is indicative of a patient with susceptibility to development of diseases related to EC-SOD deficiency. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-EC-SOD antibodies of the invention are immobilized onto a selected surface exhibiting protein affinity, such as a well in a polystyrene microtiter plate. Then, a test composition suspected of containing the wild-type and/or mutant EC-SOD protein antigen, such as a clinical sample, is added to the wells. After binding and/or washing to remove non-specifically bound immune complexes, the bound wild-type and/or mutant EC-SOD protein antigen may be detected. Detection is generally achieved by the addition of another anti-EC-SOD antibody that is linked to a detectable label. This type of ELISA is a simple “sandwich ELISA”. Detection may also be achieved by the addition of a second anti-EC-SOD antibody, followed by the addition of a third antibody that has binding affinity for the second antibody, with the third antibody being linked to a detectable label.

In another exemplary ELISA, the samples suspected of containing the wild-type and/or mutant EC-SOD protein antigen are immobilized onto the well surface and/or then contacted with the anti-EC-SOD antibodies of the invention. After binding and/or washing to remove non-specifically bound immune complexes, the bound anti-EC-SOD antibodies are detected. Where the initial anti-EC-SOD antibodies are linked to a detectable label, the immune complexes may be detected directly. Again, the immune complexes may be detected using a second antibody that has binding affinity for the first anti-EC-SOD antibody, with the second antibody being linked to a detectable label.

Another ELISA in which the wild-type and/or mutant EC-SOD proteins, polypeptides and/or peptides are immobilized, involves the use of antibody competition in the detection. In this ELISA, labeled antibodies against wild-type or mutant EC-SOD protein are added to the wells, allowed to bind, and/or detected by means of their label. The amount of wild-type or mutant EC-SOD protein antigen in an unknown sample is then determined by mixing the sample with the labeled antibodies against wild-type and/or mutant EC-SOD before and/or during incubation with coated wells. The presence of wild-type and/or mutant EC-SOD protein in the sample acts to reduce the amount of antibody against wild-type or mutant EC-SOD protein available for binding to the well and thus reduces the ultimate signal. This is also appropriate for detecting antibodies against wild-type or mutant EC-SOD protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing PBS-Tween).

After incubation with the labeled antibody, and subsequent to washing to remove unbound material, the amount of label is quantified, e.g., by incubation with a substrate such as urea, or bromocresol purple, or 2,2′-azino-di-(3-etylbenzthiazoline-6-sulfonic acid (ABTS), or H₂O₂, in the case of peroxidase as the enzyme label. Quantification is then achieved by measuring the degree of color generated, e.g., using a visible spectra spectrophotometer.

Measuring Plasma Level of EC-SOD

In an embodiment of the present invention there is a method of detecting susceptibility to development of diseases related to EC-SOD deficiency in an individual, comprising the steps of obtaining a plasma sample from the individual and assaying the plasma sample for EC-SOD level or activity, wherein a high plasma level of EC-SOD level or activity indicates that the individual is susceptible to development of diseases related to EC-SOD deficiency.

Various methods are known by those of skill in the art to measure plasma level of EC-SOD. For example, an ELISA assay as described above may be used to quantify levels of EC-SOD protein. Chromatography on concanavalin A Sepharose (Pharmacia Biotech) may be used to isolate EC-SOD from other forms of SOD. Unlike CuZn SOD and Mn SOD, the glycoprotein in EC SOD binds to the lectin concanavalin A. An ELISA is performed using primary antibodies (e.g., a rabbit polyclonal antibody against human EC-SOD and secondary antibodies (e.g., a goat, anti-rabbit antibody labeled with a detectable label such as horseradish peroxidase).

A superoxide dismutase activity assay kit (e.g., Cat. No. APT290 from CHEMICON® International Inc.) may be used to determine the level of superoxide dismutase activity in the plasma. In the assay; superoxide anions (O₂⁻.) are generated by a Xanthine/Xanthine Oxidase (XOD) system and detected by a Chromagen Solution. When SOD is present, superoxide concentrations are lowered, thereby lowering the colorimetric signal. Samples can be compared against an SOD standard for quantitative measurement or evaluated qualitatively.

A Superoxide Dismutase Standard Curve is generated first by thawing Superoxide Dismutase Standard at 2-8° C. A dilution series of Superoxide Dismutase Standard is made in the concentration range of 0.06-4 Units/μL by diluting a stock solution in assay buffer. Next, 10 μL of each dilution is transferred to a 96-well microtiter plate. Assay Buffer is included as a blank. The following components are then combined into a master mixture (adjusted according to the number of wells needed): Xanthine Solution, Chromagen Solution, 10× SOD Assay Buffer and deionized water. The master mixture is then added to each well. Finally, Xanthine Oxidase is added to each well to initiate the reaction, and the components in the well are mixed and incubated for 1-2 hours at 37 C. Optical density is measured at 490 nm on a microplate reader.

Plasma samples are assayed for SOD activity by combining the plasma sample, Xanthine Solution, Chromagen Solution, 10× SOD Assay Buffer and deionized water. Finally, Xanthine Oxidase is added to each well to initiate the reaction, and the components in the well are mixed and incubated for 1-2 hours at 37 C. Optical density is measured at 490 nm on a microplate reader.

A luminescence assay to determine the level of superoxide dismutase activity in the plasma is also described in Example 1.

Extracellular Superoxide Dismutase and Cardiovascular and other Disease

Excessive production and/or inadequate removal of reactive oxygen species, especially superoxide anion (O₂⁻.), have been implicated in the pathogenesis of many diseases, especially cardiovascular diseases, including atherosclerosis, hypertension, diabetes, and in endothelial dysfunction by decreasing nitric oxide (NO) bioactivity. Since the vascular levels of O₂⁻. are regulated by the superoxide dismutase (SOD) enzymes, a role of SOD in the cardiovascular disease is of substantial interest. A major form of SOD in the vessel wall is EC-SOD. Studies have shown that EC-SOD has a role in the development of cardiovascular and other diseases.

Nitroxide Compounds

The “nitroxide compounds,” which may be useful in the present invention, will be structurally diverse because the requisite property of the nitroxides is their ability to mimic superoxide dismutase (SOD) and catalase activity via the nitroxide free radical. The main requirement of the nitroxide compound is the presence of a stable free radical. Therefore, the nitroxides described in this invention include stable nitroxide free radicals, their precursors, and their derivatives in a heterocyclic or linear structure, as represented by the general formula:

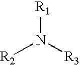

where R₁ and R₂ combine together with the nitrogen to form a heterocyclic group; and wherein the atoms in the heterocyclic group may be all carbon atoms, or may be carbon atoms as well as one or more N, O, and/or S atoms (such as, but not limited to a pyrrole, imidazole, oxazole, thiazole, pyrazole, 3-pyrroline, pyrrolidine, pyridine, pyrimidine, or purine, or derivatives thereof). The heterocyclic group is preferably a 5-membered ring (such as PROXYL, or pyrroline) or a 6-membered ring (such as piperidinyl or TEMPO), with substitution at the carbon alpha to the nitrogen by electron donating groups, which may include straight or branched chain alkyl or aryl groups, preferably methyl or ethyl groups, although other longer carbon chain species could be used.

In a more preferred embodiment, the TEMPO, DOXYL or PROXYL nitroxides or their derivatives may be used, as shown below:

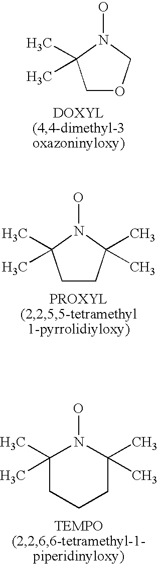

The TEMPOL, DOXYL or PROXYL nitroxides may or may not be substituted at any atom, other than the nitrogen bearing the oxygen free radical, with any combination of at least one of the following substituents: acetamido, aminomethyl, benzoyl, 2 bromoacetamido, 2-(2-(2-bromoacetamido)ethoxy) ethylcarbamoyl, carbamoyl, carboxy, cyano, 5-(dimethylamino)-1-naphthalenesulfonamido, ethoxyfluorophosphinyloxy, ethyl, 5-fluoro-2,4-dinitroanilino, hydroxy, 2-iodoacetamido, isothiocyanato, isothiocyanatomethyl, methyl, maleimido, maleimidoethyl, 2-(2maleimidoethoxy)ethylcarbamoyl, maleimidomethyl, maleimido, oxo, and phosphonooxy. The TEMPO, DOXYL or PROXYL nitroxides may also be substituents on, for example, 17b-hydroxy-5α-androstane, decane, nonadecane, 5α-cholestane, stearic acid. In the alternative, the TEMPO, DOXYL or PROXYL nitroxides may form the methyl, ethyl, or propyl ester with stearic acid. Additional nitroxides that are within the scope of the present invention are discussed in U.S. Pat. Nos. 5,462,946 and 5,591,710.

The most preferred embodiment of the invention are the nitroxides, 4-hydroxy-2,2,6,6-tetramethyl-1 piperidinyloxy (TEMPOL) or less preferred, “4-amino-tempo” (4-amino-2,2,6,6-tetramethylpiperidine-1-oxyl) or “3-CP” (3-carbomoyl-proxyl).

Nitroxide SOD Mimetics

The nitroxide SOD mimetic compounds employed in the method of the present invention will comprise a non-proteinaceous catalyst for the dismutation of superoxide anions (“nitroxide SOD mimetic”) as opposed to a native form of the SOD enzyme. As utilized herein, the term “nitroxide SOD mimetic” means a low-molecular-weight catalyst for the conversion of superoxide anions into hydrogen peroxide and molecular oxygen. Nitroxide SOD mimetics are generally preferred for use in the method of the present invention because of the limitations associated with native SOD therapies such as, solution instability, limited cellular accessibility due to their size, immunogenicity, bell-shaped dose response curves, short half-lives, costs of production, and proteolytic digestion. For example, the native SOD, CuZn, has a molecular weight of 33,000 kD. In contrast, nitroxide SOD mimetics have an approximate molecular weight of 500 to 600 kD.

Oxidative stress implies that reactive oxygen species, including superoxide anion (O₂⁻.), are produced in excess of their metabolism. Defense is provided primarily by SOD that metabolizes O₂⁻. to H₂0₂ and by catalase and glutathione peroxidase that metabolize H₂0₂ to water and oxygen. O₂⁻. enhances the contractility of blood vessels during stimulation with agonists, such as angiotensin II (ANG II). O₂⁻. may cause hypertension by many mechanisms, including bioinactivation of nitric oxide (NO), by central actions, by enhancing the peripheral sympathetic nervous system, or by enhancing renal tubular NaCl reabsorption. Oxidative stress accompanies hypertension in many models of hypertension, including the spontaneously hypertensive rat (SHR). Investigators have shown that tempol (T) is a permeant amphipathic radical nitroxide (N) that detoxifies oxygen metabolites by redox cycling through one-electron transfer reactions. The nitroxide/oxoammonium cation pair form an efficient redox coupling that mimics the enzymic action of SOD and confers catalase-like action to heme proteins. Although T lowers blood pressure (BP) in many animal models of hypertension accompanied by oxidative stress, including the SHR, the mechanisms of its in vivo action are not clearly established.

Other investigators have shown that T given to deoxycorticosterone acetate-salt rats reduces BP before it has dissipated O₂⁻. in the aorta. This acute antihypertensive response is accompanied by reduced renal sympathetic nervous system activity. It is unclear whether these neural actions of T depend on SOD mimetic action. Nevertheless, intravenous injection of liposomal (L), polyethylene-glycol, or heparin-bonded SOD lowers BP in SHR or ANG-11-infused hypertensive rats or restores acetylcholine (Ach)-induced relaxation in blood vessels from atherosclerotic rabbits.

Investigators have studied the hypothesis that the acute antihypertensive response to radical Ns is determined by their chemical class, SOD mimetic activity, or lipophilicity (Patel K et al. 2006 Am J Physiol Regul Integr Comp Physiol 290:R37-R43). These studies were conducted in anesthetized SHR because this model has a robust acute antihypertensive response to T. The acute in vivo dose-response relationships for a family of radical Ns were related to in vitro measurements of SOD mimetic activity and lipid solubility and were compared with native and liposomal (L)-Cu/Zn SOD.

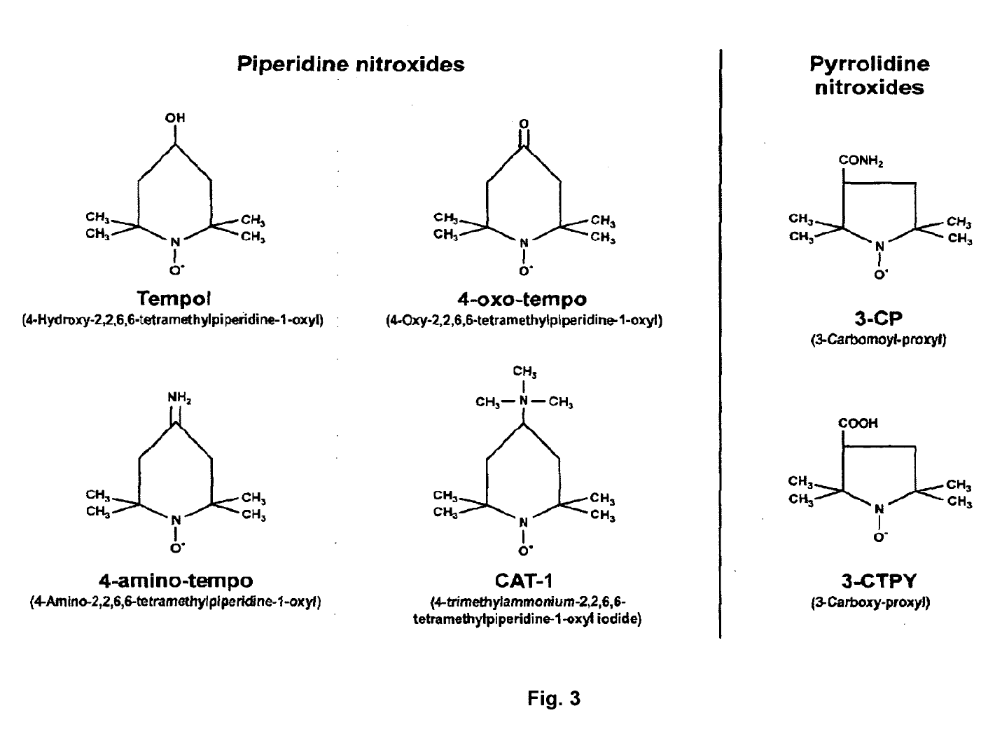

Experiments were approved by the Georgetown University Animal Care and Use Committee. Male SHR weighing 250-350 g (Taconic, Germantown, N.Y.) were anesthetized with thiobutabarbital (Inactin; Sigma, St. Louis, Mo.) (10 mg/100 g) after halothane (Halocarbon Laboratories, River Edge, N.J.) induction. Rats received 0.9% NaCl at 2 ml/h IV up to the time of receiving drugs to maintain euvolemia. A femoral artery was cannulated with polyethelene (PE)-50 tubing connected to a digital blood pressure analyzer (Micro-Med, Louisville, Ky.). Animals were equilibrated for 45 min. Thereafter, a nitroxide (17 μmol/kg) was infused intravenously over 10 s. The mean arterial pressure (MAP) and heart rate (HR) were recorded over the first 5 min and at 10 and 15 min. This was followed by doses of 54, 72, 174, and 270 μmol/kg under similar conditions. The same protocol was followed with each nitroxide: T, 4-oxo-tempo (OT), 4-amino-tempo (AT), 3-carboxy-peroxyl (3-CTPY) (Aldrich, Milwaukee, Wis.), 3-carbomyl-proxyl (3-CP) (Sigma), and 4-trimethylammonium-2,2,6,6,-tetramethylpiperidine-1-oxyl iodide (CAT-1) (Molecular Probes, Eugene, Oreg.) (FIG. 3). Doses of bovine Cu/Zn SOD (MW 32,500; Oxis Research, Portland, Oreg.) were selected that had equivalent SOD mimetic activity in vitro to the doses of T used for intravenous studies: 34, 110, 140, 260, 350, and 540 U/kg. SOD injection followed a similar protocol to the nitroxides and was compared with vehicle-injected rats. Additional rats received injections of previously boiled SOD as a further control group. Six rats were studied in each group.

The SOD activities of Ns were evaluated in vitro for their efficacy in dampening O₂⁻. generated by xanthine (25 μmol/l) plus xanthine oxidase (9 IU/ml). Their lipid solubility was assessed by shaking the Ns in a 50:50 mixture of PBS (Ambion, Austin, Tex.) and chloroform (CHCl₃; EM Science, Gibbstown, N.J.), taking an aliquot of the PBS phase, evaporating the CHCl₃phase to dryness, reconstituting it in PBS, and assessing the SOD activities in the two solutions. O₂⁻. was assessed by chemiluminescence with 5 μM lucigenin (AutoLumatPlus LB 953; EG&G, Berthold, Germany). The reduction in the stable peak value for chemiluminescence for each N, relative to vehicle, defined the SOD mimetic activity.

The mean±SE changes in MAP and HR were calculated from the individual dose-response relations. The responsiveness was the maximum effect, and the sensitivity was the expected dose for ED₅₀. Data were analyzed by ANOVA with post hoc testing by the Dunnett test, where appropriate. Statistical significance was taken as P<0.05.

The results were as follows. The body weight and baseline values for MAP and HR did not differ significantly between groups. The maximum reductions in MAP and HR during graded intravenous T were apparent within 1 min. This was followed, at lower doses, by a return to baseline over 15 min or an incomplete return at higher doses. AT, OT, and CAT-1 also caused graded reductions in MAP and HR, whereas 3-CP, 3-CTPY, and vehicle were ineffective. There was a greater maximal fall in MAP with T than CAT-1.

The time course of changes in MAP during graded intravenous doses of native Cu/Zn SOD were analyzed. Unlike T, there was no acute effect with SOD. However, the MAP became lower in SHR given Cu/Zn SOD than rats given vehicle, but this was delayed 90-110 min. The response to liposomal Cu/Zn SOD was strictly comparable to native Cu/Zn SOD and followed a similar time course. BP responses to previously boiled SOD did not differ from vehicle.

The piperidines, T, AT, OT, and CAT-1 decreased MAP and HR acutely, whereas the pyrrolidines, 3-CP and 3-CTPY, and SOD and L-SOD did not. T and AT caused the greatest reductions in MAP, whereas OT and CAT-1 caused significantly more modest reductions. T, AT, and CAT-1 caused similar reductions in HR, whereas the reduction with OT was smaller. In contrast, the charged cationic CAT-1 had the lowest ED₅₀ (greatest sensitivity) for changes in both MAP and HR, whereas the basic, lipophilic AT and OT had the highest ED₅₀s.

SOD activity by the nitroxides was assessed in vitro (Table 1). When tested in PBS, T and AT at 10⁻⁴ M extinguished 92 and 88%, respectively, of O₂⁻. generated by a xanthine-xanthine oxidase reaction, whereas 3-CP, OT, CAT1, and 3-CTPY extinguished a significantly lower fraction. The ratio of the activity that partitioned into CHCl₃ compared with PBS was examined. OT demonstrated the greatest partition ratio, indicating strong lipophilicity. CAT-1 and 3-CP demonstrated ratios <1, indicating strong hydrophilicity.

TABLE 1
Inhibition of O2-• Generated by
Xanthine-Xanthine Oxidase in vitro

| | | Maximum inhibition, | ED₅₀, |
|---|---|---|---|
| Compound | Number of Studies | % | μmol/kg |
| T | 5 | 92 ± 1 | 6 ± 1 |
| AT | 5 | 88 ± 2† | 6 ± 4 |
| 3-CP | 5 | 82 ± 1† | 18 ± 3* |
| OT | 5 | 62 ± 2† | 48 ± 7† |
| CAT-1 | 5 | 42 ± 2† | 47 ± 8† |
| 3-CTPY | 5 | 15 ± 3† | ∞† |

Values are presented as means ± SE; compared with T:
*P < 0.01;
†P < 0.005.

The group mean relationships between the antihypertensive responsiveness and the SOD mimetic activity among the effective piperidine nitroxides was analyzed. The antihypertensive ED₅₀ and the partition coefficient for the four piperidine nitroxides was also examined. The maximum change in MAP was correlated inversely with SOD activity (r=−0.94; P<0.02), whereas the ED₅₀ was correlated with the partition coefficient (r=0.89; P<0.05).

These findings confirm that T reduces BP and HR in the SHR. The main new findings are that the acute group mean antihypertensive response to piperidine nitroxides is predicted by their SOD mimetic activity, whereas the group mean ED₅₀ is predicted by their lipophilicity. Pyrrolidine nitroxides do not reduce MAP, despite possessing in vitro SOD activity. Neither native nor liposomal SOD reduces MAP acutely but both cause similar falls in MAP, albeit less than T, that are delayed 90-110 min.

Highly polar compounds that do not penetrate cells have a relatively restricted peak volume of distribution, leading to higher initial plasma levels. This can explain the dependence of the antihypertensive sensitivity of the piperidine nitroxides on their hydrophilicity.

Both five-member ring pyrrolidine nitroxides and six-member ring piperidine nitroxides acted as SOD mimetics in this in vitro assay. Although 3-CP is a very effective SOD mimetic in vitro, this study confirms that it does not reduce BP in vivo. The data in this study are consistent with redox chemistry of nitroxides. Electron paramagnetic resonance spectrometry, cyclic voltammetry, and bulk electrolysis has been used to characterize and quantitate the redox midpoint potential of nitroxides. Among the six-member ring nitroxides, their antihypertensive effect in this study follows their redox midpoint potentials (E_{1/2}) measured previously in the literature. Thus the lower E_{1/2} of the six-member ring nitroxide, the better its SOD mimetic capability and the more effective it was in reducing the BP. For example, T and 4-aminotempol have E_{1/2} values of 800 and 820 mV, respectively, whereas oxotempol has a higher E_{1/2} of 913 mV, and CAT-1, although not estimated in the prior published studies, has significantly higher values still. Thus the E_{1/2} values follow the rank order of antihypertensive activities of the four components tested. It is established that six-member ring nitroxides participate in redox reactions more effectively and rapidly than five-member rings because their reversible conformational transformation between “boat” and “chair” structures facilitates access to reactants, making them kinetically more effective, in addition to thermodynamic considerations. It is during interconversion between “boat” and “chair” configuration that the NO site on the six-member nitroxide is exposed for catalysis. In contrast, five-member rings are always planar and thus less reactive.

The antihypertensive response to the piperidine nitroxides was independent of their lipophilicity. CAT-1 is a highly polar tetramethylammonium compound that is as effective (relative to SOD activity) in lowering MAP in vivo as the highly lipophilic compounds, 4-oxo-tempo or T. Studies in mice with gene deletions of Cu/Zn or extracellular (EC)-SOD or given SOD inhibitors have concluded that vascular NO is protected by SOD from bioinactivation by O₂⁻., both intracellularly and extracellularly. Diffusional cellular uptake depends on lipophilicity. Therefore, the finding that the acute antihypertensive response to nitroxides is independent of lipophilicity suggests that the initial effects are extracellular, Indeed, CAT-1 was effective in reducing BP acutely. CAT-1 does not permeate cells but can react with cell membrane components.

Therefore, the hypothesis that the acute antihypertensive response to SOD does not require cellular uptake by the use of native and liposome-encapsulated Cu/Zn SOD was investigated. Neither had any acute antihypertensive action that caused a similar, moderate, and delayed reduction in BP over 110 min. These findings are consistent with previous reports. Recombinant EC-SOD injected into wild-type mice infused with ANG II has no immediate effect, although there is a fall in BP in EC-SOD knockout mice. Liposomal Cu/Zn SOD injected intravenously over 5 days into cholesterol-fed rabbits with atherosclerosis is taken up in both endothelial and vascular smooth muscle cells, where it increases SOD activity and partially restores endothelium-dependent relaxation to ACh. Liposomal Cu/Zn SOD given over 5 days reduces the MAP and improves ACh-induced vascular relaxations in rats infused with ANG II, but not with norepinephrine. Heparin-bonded SOD binds to endothelial cells, penetrates extravascularly, and causes a delayed lowering of MAP after intravenous injection, whereas native SOD is excluded and does not acutely reduce the MAP. Injection of polyethylene-glycolated SOD for 1 week into cholesterol-fed, atherosclerotic rabbits increases blood vessel SOD activity and partially restores endothelium-dependent relaxation to ACh. Because liposomal SOD is taken up into endothelial cells, the similar MAP responses to native and liposomal Cu/Zn SOD in the present study suggest that the antihypertensive action of SOD is not exerted in the vascular endothelium. The slower response to SOD may relate to its larger molecular size compared with nitroxides. This suggests that SOD must diffuse from the vascular space into the interstitium to lower BP. Native SOD has a plasma half time of 6 min after intravenous administration, with ˜10% of injected SOD being associated with the kidney 45 min after injection.

Piperidine nitroxides exert an acute combination of a rapid, substantial, and reversible reduction in MAP, accompanied by bradycardia. These characteristics could make piperidine nitroxides ideal agents for the treatment of hypertensive crises. The reduction in HR and sympathetic nerve tone after intravenous nitroxides could give these compounds an advantage over sodium nitroprusside, which causes reflex tachycardia and cardiac stimulation. The finding that the effectiveness of nitroxides is predicted by their SOD mimetic activity provides a rational basis for selection of T or AT for this indication. T is also effective as an oral hypertensive antioxidant agent in the SHR model. In the present invention, the selection of nitroxides is based upon their SOD mimetic activity, for which many different structure-function are described by Patel et al., 2006.

Tempol is a well-validated spin trap for O₂⁻.(Denzlinger C et al. 1991 Br J Pharmacol 102:865-870; Krishna C M et al. 1996 J Biol Chem 271:26026-26031). It permeates cell membranes freely and acts catalytically to metabolize O₂⁻. to H₂O₂ (Konorev E A et al. 1995 Free Rad Biol Med 18:169-177; Krishna C M et al. 1996 J Biol Chem 271:26026-26031; Mitchell J B et al. 1990 Biochem 29:2802-2807). It further facilitates metabolism of H₂O₂ to O₂ and H₂O (Krishna C M et al. 1996 J Biol Chem 271:26018-26025). Tempol protects cells or tissues from damage due to oxidative stress accompanying cardiac ischemia (Gelvan D et al. 1991 Proc Natl Acad Sci USA 88:4680-4684), colitis (Karmeli F et al. 1995 Gut 37:386-393), hyperoxia (Mitchell J B et al. 1991 Arch Biochem Biophys 289:62-70), or radiation (Hahn S M et al. 1992 Cancer Res 52:1750-1753).

Administration

The compounds of the invention include nitroxide compounds that can be administered via either the oral, parenteral or topical routes and other routes of administration known to those skilled in the art. In general, these compounds are most desirably administered in the dosages discussed as described below, although variations will necessarily occur depending upon the weight, age, and condition of the subject being treated and the presence of co-morbid conditions that may affect the pharmokokinetics or pharmokodynamics of the agents. These will vary according to the particular route of administration chosen. Other variations may also occur depending upon the species of animal being treated and its individual response to said medicament, as well as on the type of pharmaceutical formulation chosen, and the time period and interval at which such administration is carried out. In some instances, dosage levels below the lower limit of a range may be more than adequate, while in other cases still larger doses may be employed without causing any harmful side effects, provided that such larger doses are first divided into several small doses for administration throughout the day or via sustained release formulations, or by continuous administration by intravenous infusion or dermal application. For example, tablets may be uncoated or they may be coated by known techniques to delay disintegration and absorption in the gastrointestinal tract thereby providing a sustained action over a longer period. Potential time delayed materials include glyceryl monostearate or glyceryl distearate. They may also be coated by the techniques described in the literature to form osmotic therapeutic tablets for control release.

The compounds of the invention may be administered alone or in combination with pharmaceutically acceptable carriers of diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored.

For oral administration, tablets containing various excipients such as microcrystalline cellulose, sodium citrate, calcium carbonate, dicalcium phosphate and glycine may be employed along with various disintegrants such as starch (e.g., preferably corn, potato or tapioca starch), alginic acid and certain complex silicates, together with granulation binders such as polyvinylpyrrolidone, sucrose, gelatin, and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar, as well as high molecular weight polyethylene glycols.

When aqueous suspensions and/or elixirs are desired for oral administration, the active ingredient may be combined with various sweetening or flavoring agents, coloring matters or dyes, and, if so desired, emulsifying and/or suspending agents as well, together with such diluents as water, ethanol, propylene glycol, glycerin and various like combinations thereof. Aqueous suspensions may also contain the active materials in admixture with excipients suitable for aqueous suspensions. Useful suspending agents include, for example, sodium carboxymethyl cellulose, methylcellulose, hydroxypropylmethylcellulose, sodium alginate, polyvinylpyrrolidone, gum tragacanth and gum acacia; dispersing or wetting agents may be a naturally occurring phosphatide, for example, lecithin, or condensation products of an alkylene oxide with fatty acids, for example, lecithin or condensation products of an alkylene oxide with fatty acids (e.g., polyoxyethylene stearate), or condensation products of ethylene oxide with long chain aliphatic alcohols (e.g., heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyoxyethylene sorbitan monooleate). The aqueous suspensions may also contain one or more preservatives (e.g., ethyl or n-propyl p-hydroxybenzoate).

For parenteral administration, solutions of a therapeutic compound of the present invention could be formulated as a ready to use solution in an isotonic vehicle of normal saline containing suitable stabilizers. The active agent may also be formulated as a dry, sterile powder or as a lyophilized powder which would require reconstitution with an acceptable isotonic, sterile liquid. These aqueous solutions are suitable for intravenous, intramuscular, or subcutaneous injection purposes. The preparation of all these solutions under sterile conditions is readily accomplished by standard pharmaceutical techniques well known to those skilled in the art.

Pharmaceutical Compositions

The following compositions are suggestions only and are not meant to limit the scope of the invention. Oral compositions may contain fillers and, additionally, preservatives along with other inert or active agents.

Compositions for Oral Administration: 500 mg Tempol, 500 mg Starch, 5 mg Magnesium Stearate.

The composition may be placed in capsules which may be enteric coated. Other preparations can include concentrations of Tempol from about 0.01 mg/kg/day to about 500 mg/kg/day. In rats, effective dosages administered orally include from about 0.7 to about 15,000 mg/kg/day orally, or more preferred from about 0.7 to about 1,500 mg/kg/day orally, or most preferred from about 7 to about 150 mg/kg/day. In humans, because of the slower metabolism, the effective dosages of Tempol administered orally include from about 0.07 to about 7,500 mg/kg/day orally, or more preferred from about 0.07 to about 750 mg/kg/day orally, or most preferred from about 0.7 to about 75 mg/kg/day.

Compositions for Parenteral Administration

From about 1 gram of Tempol is added to from about 5.0 to about 100 mls of 5% dextrose or normal saline or other suitable isotonic solution for intravenous (i.v.) administration. Additional compositions contemplated for parenteral use include from about 0.5 mM to about 100 mM Tempol. More preferred would be about 0.5 mM to about 10 mM Tempol administered in an isotonic vehicle intravenously (i. v.).

Compositions for Intravenous Administration

In rats, effective dosages administered intravenously (i.v.) include: (1) from about 0.25 to about 800 mg/kg by i.v. bolus dosing, more preferred from about 0.25 to about 80 mg/kg by i.v. bolus dosing, and most preferred from about 2.5 mg/kg to about 8 mg/kg by i.v. bolus dosing; and (2) from about 0.5 to about 2,000 mg/kg/hr by i. v. infusion, more preferred from about 0.5 to about 200 mg/kg/hour by i. v. infusion, and most preferred from about 5 to about 20 mg/kg/hour by i. v. infusion. In humans, because of the slower metabolism, the effective dosages of Tempol administered intravenously include: (1) from about 0.025 to about 400 mg/kg by i.v. bolus dosing, more preferred from about 0.025 to about 40 mg/kg by i.v. bolus dosing, and most preferred from about 0.25 mg/kg to about 4 mg/kg by i.v. bolus dosing; and (2) from about 0.05 to about 1,000 mg/kg/hr by i.v. infusion, more preferred from about 0.05 to about 100 mg/kg/hour by i. v. infusion, and most preferred from about 0.5 to about 10 mg/kg/hour by i. v. infusion.

Compositions for Dermal Administration

Tempol may be administered on a solid support. One example of a solid support are patches. Patches for the administration of Tempol can be formulated as adhesive patches containing a nitroxide. For example, the patch may be a discoid in which a pressure-sensitive silicone adhesive matrix containing the active agent may be covered with a non-permeable backing. The discoid may either contain the active agent in the adhesive or may have attached thereto a support made of material such as polyurethane foam or gauze that will hold the active agent (e.g., Tempol). Before use, the material containing the active agent would be covered to protect the patch.

A patch or other solid support composed of trilarninate of an adhesive matrix sandwiched between a non-permeable backing and a protective covering layer is prepared in the following manner: Two grams of Tempol is applied to from about 5 grams of a pressure sensitive silicone adhesive composition BIOPSA™ Q7-2920 (Dow Corning Corp., Midland, Mich., U.S.A.). The adhesive is applied to a polyester film to provide in successive layers about 200 mg of active agent per cm². The film containing the adhesive is then made into a patch of 10 cm². The patch is covered with a protective layer to be removed before application of the patch.

Patches may be prepared containing permeation enhancers such as cyclodextrin, butylated hydroxyanisole, or butylated hydroxytoluene. However, it should be remembered that the active agents of this invention are effective on application to the epidermal tissue. When the patches are to be applied to thin or abraded skin, there is little need to add a permeation enhancer.

### Role of Extracellular Superoxide Dismutase in the Mouse Angiotensin Slow Pressor Response

Low rates of angiotensin II (Ang II) infusion raise blood pressure, renal vascular resistance (RVR), NADPH oxidase activity and superoxide. We tested the hypothesis that these effects are ameliorated by extracellular superoxide dismutase (EC-SOD). EC-SOD knockout (−/−) and wild type (+/+) mice were equipped with blood pressure telemeters and infused subcutaneously with Ang II (400 ng/kg per minute) or vehicle for 2 weeks. During vehicle infusion, EC-SOD −/−mice had significantly (P<0.05) higher MAP (+/+: 107±3 mm Hg versus −/−: 114±2 mm Hg; n=1 1 to 14), RVR, lipid peroxidation, renal cortical p22^{phox} expression, and NADPH oxidase activity. Ang II infusion in EC-SOD +/+mice significantly (P<0.05) increased MAP, RVR, p22^{phox}, NADPH oxidase activity, and lipid peroxidation. Ang II reduced SOD activity in plasma, aorta and kidney accompanied by reduced renal EC-SOD expression. During Ang II infusion, both groups had similar values for MAP (+/+Ang II: 125±3 versus −/−Ang II: 124±3 mm Hg; P value not significant), RVR, NADPH oxidase activity, and lipid peroxidation. SOD activity in the kidneys of Ang II-infused mice was paradoxically higher in EC-SOD −/−mice (+/+: 8.8±1.2 U/mg protein⁻¹ versus −/−: 13.7±1.6 U/mg protein⁻¹; P<0.05) accompanied by a significant upregulation of mRNA and protein for Cu/Zn-SOD. In conclusion, EC-SOD protects normal mice against oxidative stress by attenuating renal p22^{phox} expression, NADPH oxidase activation, and the accompanying renal vasoconstriction and hypertension. However, during an Ang II slow pressor response, renal EC-SOD expression is reduced and, in its absence, renal Cu/Zn-SOD is upregulated and may prevent excessive Ang II-induced renal oxidative stress, renal vasoconstriction and hypertension.

An increase in reactive oxygen species (ROS) in the blood vessels and kidneys is reported in several experimental animal models of hypertension and in human essential and renovascular hypertension (Wilcox, C S and Ernest H 2005 Am J Physiol Regul Integr Comp Physiol 289:R913-R935). Infusions of angiotensin II (Ang II) increase blood pressure (BP), markers of oxidative stress, and renal expression of the p22^{phox} and Nox-1 components of renal NADPH oxidase (Chabrashvili T et al. 2003 Am J Physiol 285:R117-R124). These effects seem specific for Ang II because similar pressor infusions of norepinephrine into rats do not induce oxidative stress in blood vessels (Laursen J B et al. 1997 Circulation 95:588-593). An increased production of superoxide (O₂⁻) reduces bioactive NO (Rubanyi G M and Vanhoutte P M 1986 Am J Physiol 250:H822-H827) and contributes to vascular and renal injury in chronic hypertension (Fukai T et al. 2002 Cardiovasc Res 55:239-249; Rajagopalan S et al. 1996 J Clin Invest 97:1916-1923). Superoxide dismutase (SOD) metabolizes O₂.⁻ to H₂O₂ which is further metabolized to inactive products by peroxidases. Hypertension can be moderated or prevented by gene transfer of extracellular (EC)-SOD (Chu Y et al. 2003 Circ Res 92:461-468) or by administration of tempol (Welch W J et al. 2005 Kidney Int 68:179-187) which is a nitroxide SOD mimetic.

The 3 isoforms of SOD are localized to the kidney (Chabrashvili T et al. 2002 Hypertens 39:269-274). EC-SOD is located on cell membranes of endothelial cells and vascular smooth muscle cells (Ookawara T et al. 1998 Am J Physiol 275:C840-C847). EC-SOD −/−mice have endothelial dysfunction in conduit blood vessels that is ascribed to an impaired NO bioavailability (Jung O et al. 2003 Circ Res 93:622-629). EC-SOD expression in blood vessels is increased during pressor infusions of Ang II and may thereby limit the increase in vascular O₂⁻ (Fukai T et al. 1999 Circ Res 85:23-28). This may explain the finding that EC-SOD −/−mice have an exaggerated early increase in BP during pressor infusions of Ang II (Jung O et al. 2003 Circ Res 93:622-629). Ang II infusions at initially subpressor rates leads to a slow development of hypertension and renal vasoconstriction that depends on O₂.⁻ because these effects are prevented by tempol (Kawada N, et al. 2002 J Am Soc Nephrol 13 :2860-2868). This slow pressor response likely entails a renal mechanism because the hypertension depends on salt intake (Csiky B and Simon G 1997 Am J Physiol 273:H1275-H1282) and is accompanied by a preferential renal vasoconstriction (Imig J D 2000 Am J Hypertens 13:810-818), enhanced renal afferent arteriolar constrictor response to Ang II (Wang D et al. 2003 J Am Soc Nephrol 14:2783-2789), and salt retention (Hall J E 1986 Am J Physiol 250:R960-R972). A slow pressor response is seen in mice (Kawada N, et al. 2002 J Am Soc Nephrol 13 :2860-2868), rats (Welch W J et al. 2005 Am J Physiol 288:H22-H28; Hu L et al. 2005 J Hypertens 16:1285-1298), dogs (Caravaggi A M et al. 1976 Circ Res 38:315-321), rabbits (Wang D et al. 2004 Circ Res 94:1436-1442) and humans (Ames R P et al. 1965 J Clin Invest 44:1171-1186). It has been considered a model of human hypertension, because it is accompanied by only modest elevations in plasma Ang II concentrations (Hu L et al. 2005 J Hypertens 16:1285-1298; Brown A J et al. 1981 Am J Physiol 241:H381-H388). The role of EC-SOD in the kidney in this model is quite unclear because renal cortical EC-SOD expression in the rat is downregulated by a 2-week infusion of Ang II at a slow pressor rate (Chabrashvili T et al. 2003 Am J Physiol 285:R117-R124). Therefore we contrasted the mean arterial pressure (MAP) and renal vascular response to a slow pressor infusion of Ang II in EC-SOD −/− and +/+mice and evaluated NADPH oxidase, total SOD activity and the mRNA and protein expression of the 3 SOD isoforms, EC-, Cu/Zn- or intracellular (IC)- and Mn-SOD, in the kidneys to test the hypothesis that EC-SOD regulates renal O₂.⁻ generation, the development of hypertension, and renal vasoconstriction in this Ang II slow pressor model.

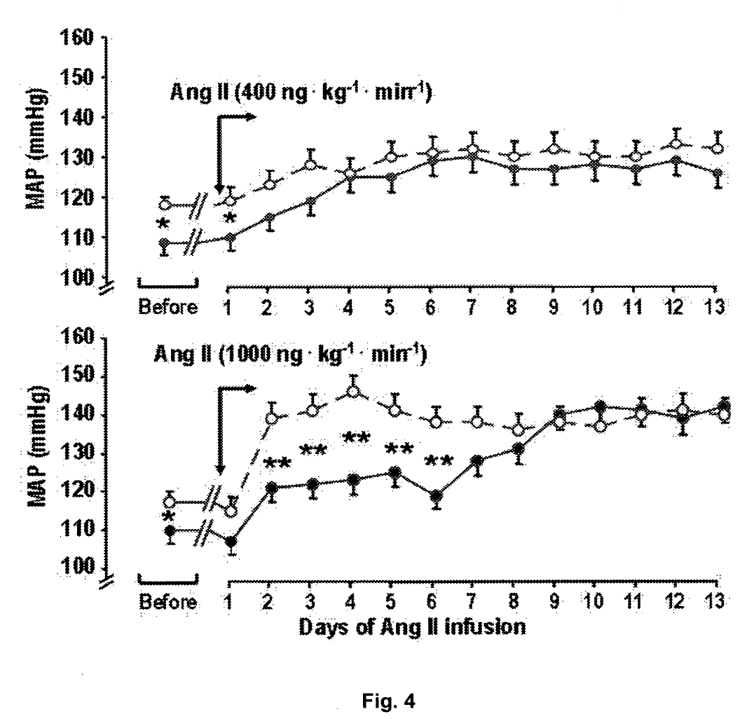

MAP of Conscious Mice: The MAP averaged over 6 control days before infusion was higher in EC-SOD −/−mice (+/+: 101±2; n=14 mm Hg versus −/−: 110±3, n=11 mm Hg; P<0.05). The MAP of both strains increased progressively during the first week of Ang II infusion at a slow pressor rate and remained elevated throughout the infusion (FIG. 4). The increase in MAP with Ang II infused at a slow pressor rate was similar in EC-SOD +/+ and −/−mice (Table 2). The MAP of mice infused with Veh did not change during the infusion. The HR was not affected by strain or infusion of Ang II. Mice infused with Ang II at a pressor rate of 1,000 ng/kg⁻¹/min⁻¹ had an abrupt increase in MAP, which was greater in EC-SOD −/− than +/+mice on days 2 to 6 of the infusion (FIG. 4) confirming a previous study (Jung O et al. 2003 Circ Res 93:622-629). However, during the second week of infusion of Ang II at the pressor rate, the MAP values became similar in EC-SOD +/+ and −/−mice. The EC-SOD −/−mice of this group had a similar increase in MAP over 10 to 13 days of Ang II infusion (Table 2). Subsequent data refer to Ang II infusion at a slow pressor rate, because the object of this study was to assess the role of EC-SOD in the slow pressor response.

TABLE 2
MAP (mmHg) of conscious and anesthetized mice: effects of strain and angiotensin II infusion

| | | | | | | |
|---|---|---|---|---|---|---|
| | | Rate of Angiotensin II | MAP Before | MAP on day 12 of | Change in MAP | Effects of Ang II, |
| Strain | Number of mice | infusion (ng · kg⁻¹ · min⁻¹) | (mmHg) | Ang II (mmHg) | with Ang II (mmHg) | p value: |

(a) Conscious mice, telemetry

| | | | | | | |
|---|---|---|---|---|---|---|
| EC-SOD (+/+) | 8 | 400 | 108.7 ± 3.8 | 134.6 ± 3.3 | +24.4 ± 3.45 | p < 0.001 |
| EC-SOD (−/−) | 9 | 400 | 117.8 ± 1.7 | 132.5 ± 4.1 | +16.7 ± 4.42 | p < 0.001 |
| p value (+/+ vs −/−) | | | p < 0.05 | ns | ns | |

(b) Anesthetized mice

| | | | | | | |
|---|---|---|---|---|---|---|
| EC-SOD (+/+) | 11 | 400 | 79.6 ± 1.9 | 92.1 ± 3.0 | — | p < 0.001 |
| EC-SOD (−/−) | 11 | 400 | 88.2 ± 2.1 | 98.4 ± 2.6 | — | p < 0.05 |
| p value (+/+ vs −/−): | | | p < 0.05 | ns | | |

(c) Conscious mice, telemetry

| | | | | | | |
|---|---|---|---|---|---|---|
| EC-SOD (+/+) | 6 | 1000 | 109.8 ± 3.1 | 149.1 ± 4.3 | +35.7 ± 6.5 | p < 0.001 |
| EC-SOD (−/−) | 6 | 1000 | 118.1 ± 2.4 | 149.9 ± 5.4 | +30.7 ± 3.3 | p < 0.001 |
| p value (+/+ vs −/−) | | | p < 0.05 | ns | ns | |

Mean ± SEM values
Before indicates mean data over 6 days before insertion of Ang II minipump.

MAP Under Anesthesia. The MAP of anesthetized, Veh-infused mice also was higher in EC-SOD −/− that in +/+mice (Table 2). By day 13 of Ang II infusion at a slow pressor rate, the MAP under anesthesia had increased to a similar value in both strains (Table 2).

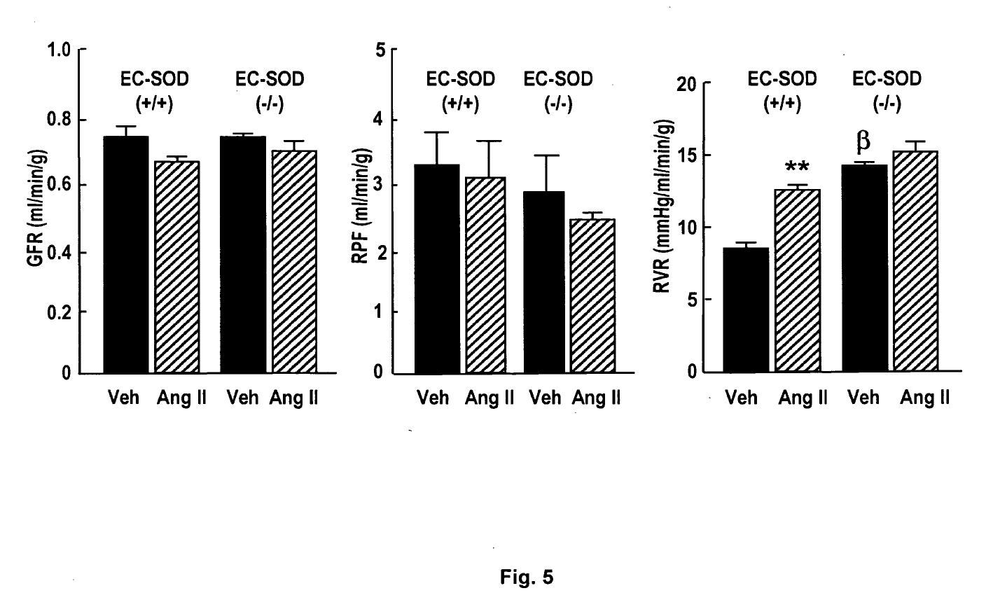

Renal Function. The glomerular filtration rate and RPF during Veh infusion were not significantly different between strains and were not significantly altered by Ang II (FIG. 5). The RVR during infusion of Veh was higher in EC-SOD −/−mice. During infusion of Ang II, RVR did not increase significantly in EC-SOD −/−mice and became comparable to values in EC-SOD +/+mice (FIG. 5).

Plasma Renin. The plasma rennin activity and plasma rennin concentration during infusion of Veh did not differ between strains. These parameters were not measured in Ang II infused mice.

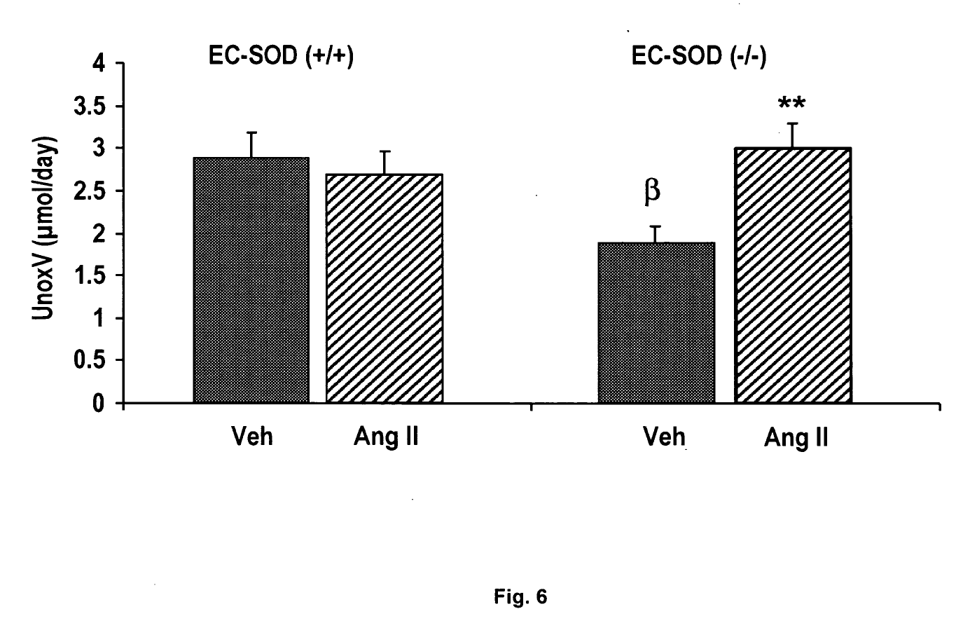

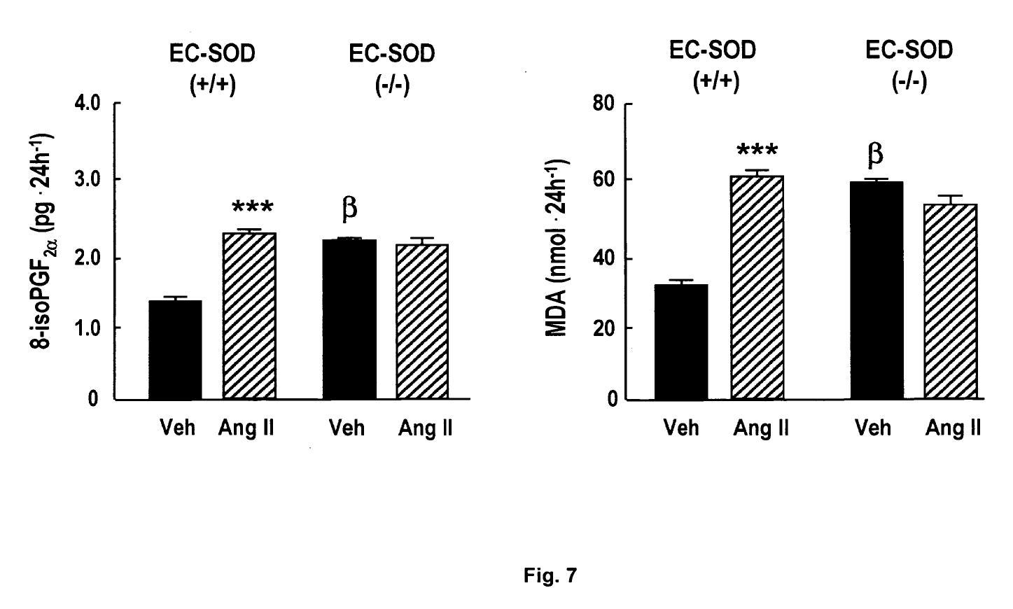

Markers of NO and Oxidative Stress. During Veh infusion, EC-SOD −/−mice had a reduced excretion of NOx, but, during Ang II, this increased only in EC-SOD −/−mice (FIG. 6). EC-SOD −/−mice had an increased excretion of 8-isoprostaglandin (+/+: 1.3±0.3; n=6 versus −/−: 2.1±0.2; n=6; pg 24⁻¹; P<0.01) and MDA (+/+: 31±3; n=6 versus −−: 59±6; n=6; nmol 24⁻¹; p<0.01; FIG. 7). Ang II increased the excretion of both markers significantly (P<0.01) in EC-SOD +/+mice, but did not change the excretion of either marker significantly in −/−mice. This resulted in similar values for NOx, 8-Isoprostaglandin, and MDA in EC-SOD −/− and +/+mice during infusion of Ang II.

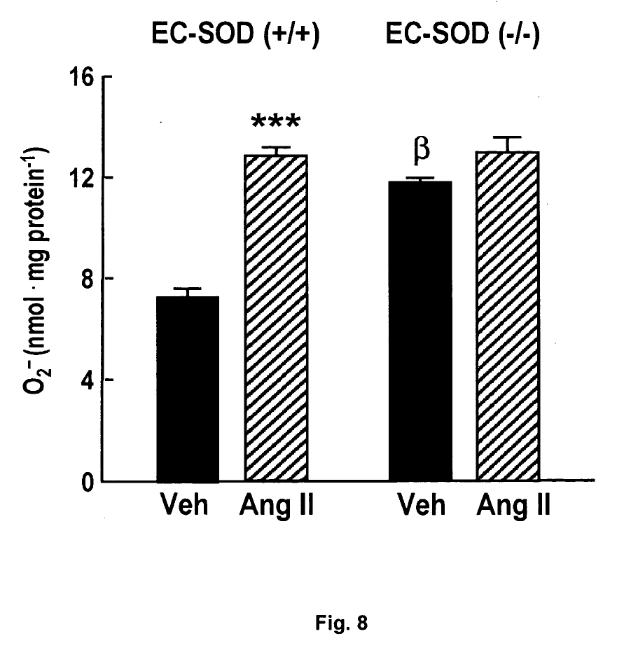

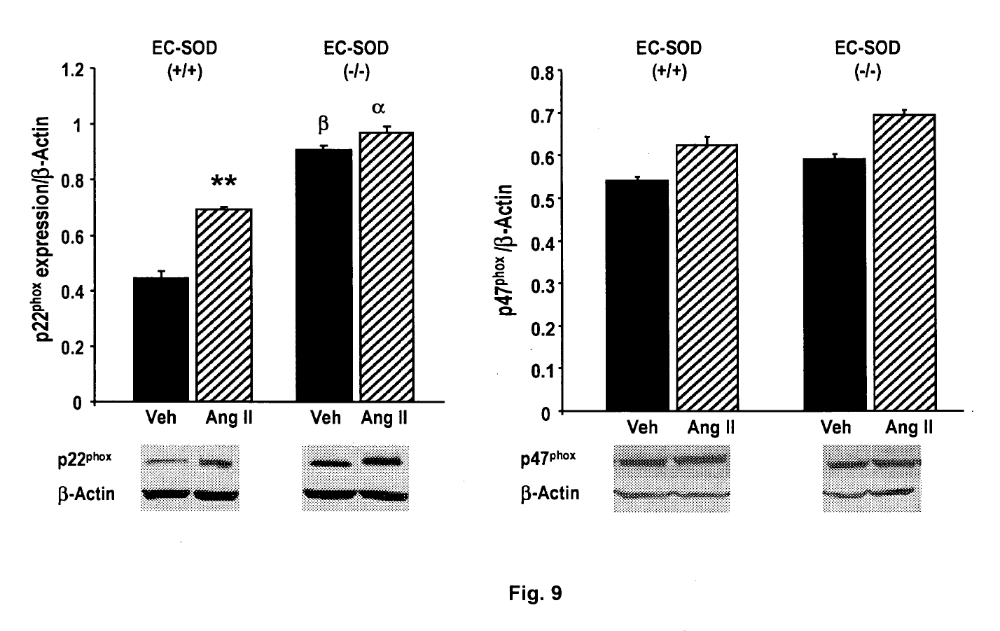

Renal NADPH-Oxidase Activity and Expression of p22^{phox} and p47^{phox}: The NADPH oxidase activity of renal cortical homogenates was greater in Veh-infused EC-SOD −/−mice (+/+: 7.2±0.9; n=6 versus −/−: 11.2±1.1; n=6; nmol/mg of protein⁻¹: <0.01; FIG. 8). This increased significantly (P<0.001) with Ang II infusion in EC-SOD +/+mice, but not in −/−mice. The renal cortical expression of p22^{phox} protein in EC-SOD −/−mice was greater (P<0.01) than in EC-SOD +/+mice during Veh infusion but increased with Ang II only in EC-SOD +/+mice (FIG. 9). There were no differences in expression of p47^{phox} (FIG. 9).

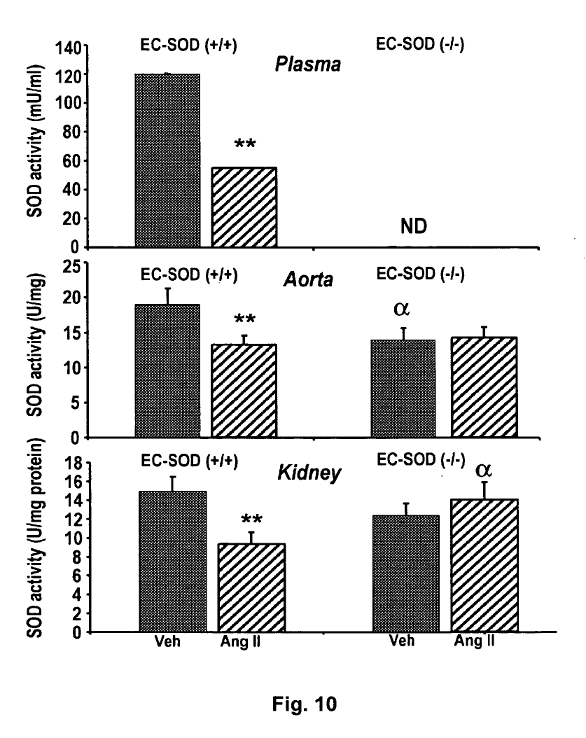

Total SOD Activity. SOD activity was not detectable in the plasma from EC-SOD −/−mice. The SOD activity in the plasma, aorta, and kidney cortex of EC-SOD +/+mice was reduced by Ang II infusion (FIG. 10). The SOD activity in the aorta of EC-SOD −/−mice was lower than in EC-SOD +/+mice during infusion of Veh (+/+: 18.2±3.2; n=6 versus −/−: 13.1±1.2; n=6; U/mg of protein⁻¹; P<0.05; FIG. 10) but was not changed significantly by Ang II. The SOD activity in the renal cortex was similar in both strains during Veh infusion (+/+Veh: 14.9±3.5; n=6 versus −/−Veh: 12.1±1.6; n=6; U/mg of protein⁻¹; P not significant; FIG. 10) and was reduced during infusion of Ang II only in EC-SOD in +/+mice, resulting in a paradoxical higher (P<0.05) SOD activity in the renal cortex of EC-SOD −/−, compared with +/+mice, during infusion of Ang II (FIG. 10).

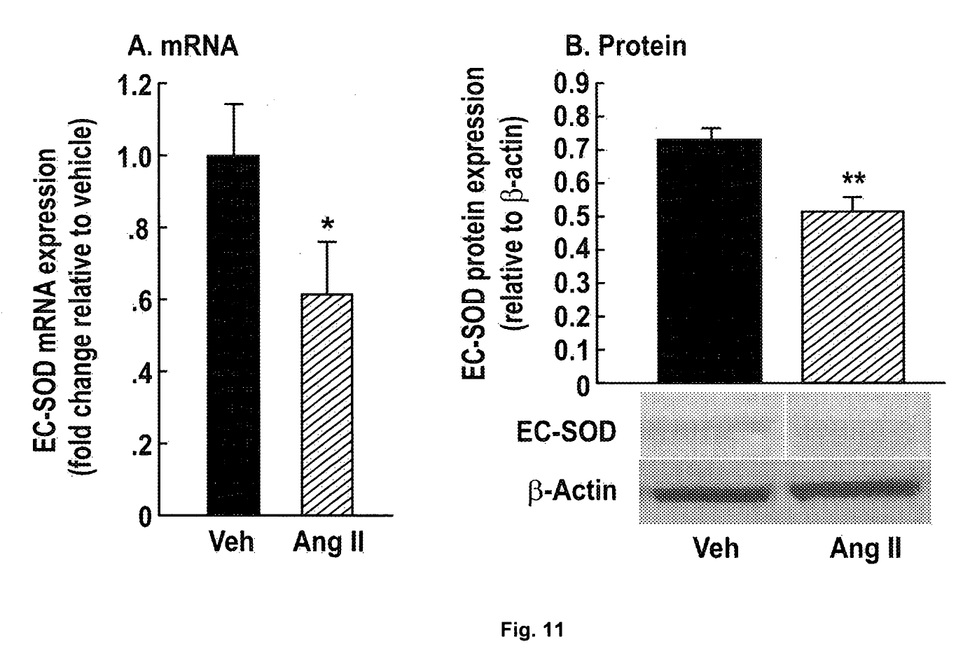

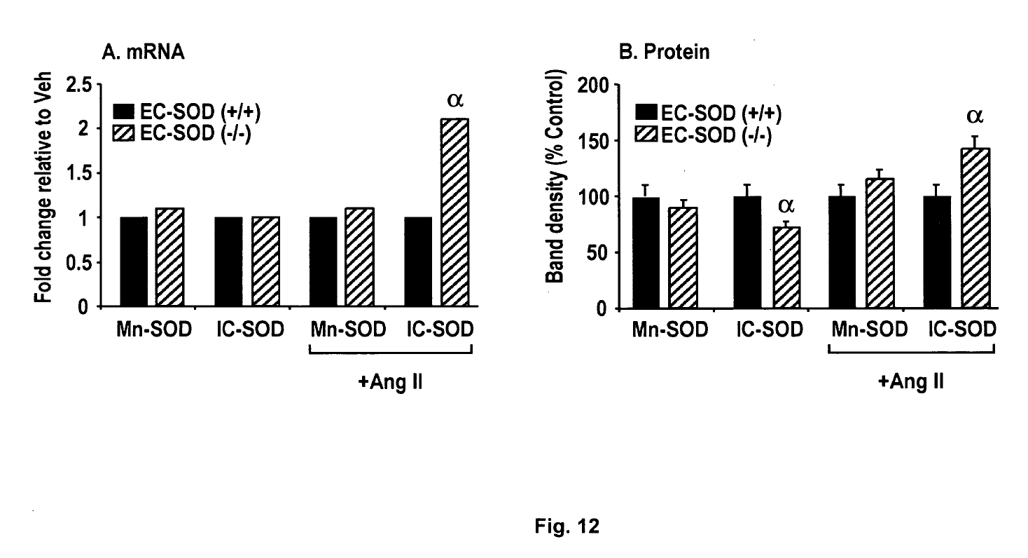

Expression of SOD Isoforms in Kidney Cortex. As anticipated, the mRNA expression for EC-SOD in the kidneys of −/−mice was not detectable. The mRNA and protein expression for EC-SOD was reduced (P<0.05) by Ang 11 in EC-SOD +/+mice, confirming our previous results in normal rats (Chabrashvili T et al. 2003 Am J Physiol 285:R1 17-R124) (FIG. 11). The mRNA and protein expression for Mn-SOD in the kidney cortex was unchanged by Ang II in either strain (FIG. 12). The expression of IC-SOD, or Cu−Zn-SOD protein, but not mRNA, in the kidney cortex of Veh-infused mice was reduced in EC-SOD −/−mice. During Ang II, the mRNA and protein expression for IC-SOD increased in EC-SOD −/−, but not in EC-SOD +/+mice and the protein concentration became higher in the kidney cortex of the EC-SOD −/−strain (FIG. 12).

Discussion

The main new findings of this study are that the MAP (whether measured in conscious mice by telemetry or in anesthetized mice by direct arterial cannulation) was modestly, but significantly, higher in EC-SOD −/−mice. This was accompanied by an increase in RVR and evidence of increased oxidative stress (increased 8-Isoprostaglandin, PGF_{2α}, and MDA excretion) and decreased NO generation (decreased NOx excretion). The increased oxidative stress in EC-SOD −/−mice was accompanied by reduced SOD activity in both the plasma and aorta, and increased p22^{phox} expression and NADPH oxidase activity in the kidney. These data indicate that the oxidative stress of EC-SOD −/−mice may originate from both a decreased metabolism of O₂.⁻ by EC-SOD and an increased generation of O₂.⁻ by NADPH oxidase. The reduced SOD activity in the aorta of EC-SOD −/−mice supports previous studies suggesting that EC-SOD is a major defense system against oxidative stress in blood vessels (Fukai T et al. 2002 Cardiovasc Res 55:239-249; Jung O et al. 2003 Circ Res 93:622-629; Stralin P et al. 1995 Arterioscler Thromb Vasc Biol 15:2032-2036). In contrast, the maintained SOD activity in the kidney cortex of SOD −/−mice, indicates that other SOD isoforms contribute to oxidative defense in the kidney.

We found that Ang II infused at a pressor rate increased the MAP more rapidly in EC-SOD −/− than +/+mice similar to previous findings (Jung O et al. 2003 Circ Res 93:622-629). In contrast, Ang II infused at a slow pressor rate led to similar increases in MAP in EC-SOD +/+ and −/−mice. The increase in excretion of lipid peroxidation products produced by the slow pressor Ang II infusion in normal mice were unexpectedly absent in EC-SOD −/−mice. This may relate to the unchanged SOD activity in the aorta and kidney, and the unchanged renal cortical expression of p22^{phox} and NADPH oxidase activity with Ang II in EC-SOD −/−mice. In contrast, the SOD activity in the plasma, aorta and kidneys of EC-SOD +/+mice was reduced, and the p22^{phox} expression and NADPH oxidase activity of the kidney cortex was increased, during Ang II infusion at a slow pressor rate. The reduced SOD activity in the aorta and kidney cortex of EC-SOD +/+mice infused with Ang II at a slow pressor rate was accompanied by a reduced mRNA and protein expression for EC-SOD, confirming previous studies in normal rats (Chabrashvili T et al. 2003 Am J Physiol 285:RI17-RI24). In contrast, the SOD activity was maintained in the aorta and actually was increased in the kidney cortex during infusion of Ang-II into EC-SOD −/−mice. This may be explained by the increased expression of the mRNA and protein for IC-SOD in the kidneys of EC-SOD −/−mice infused with Ang II.

These data suggest that 3 factors could account for the failure of Ang II infusion to increase ROS in EC-SOD −/−mice. First, Ang II infusion at a slow pressor rate reduces the expression of EC-SOD in the wild type kidney (FIG. 11). Thus, this downregulation of EC-SOD could minimize the differences in oxidative stress between EC-SOD wild-type and knockout animals during Ang II infusion. This finding that Ang II infusion at a slow pressor rate downregulates the mRNA and protein expression for EC-SOD in the kidneys of wild-type mice (FIG. 11) contrasts with the upregulation of EC-SOD expression in the aorta of rats infused with Ang-II at a pressor rate (Fukai T et al. 1999 Circ Res 85:23-28). Second, in the absence of EC-SOD, Ang II infusion up-regulates the mRNA and protein expression of IC-SOD in the kidney cortex (FIG. 12). This up-regulation of IC-SOD may be sufficient to offset any reduction in SOD activity because of the loss of EC-SOD. Third, in contrast to wild-type mice, Ang II infusion failed to increase p22^{phox} expression (FIG. 9) or NADPH oxidase activity (FIG. 8) in the kidney cortex of EC-SOD −/−mice. Collectively, these results may explain the absence of Ang II-induced increases in ROS in EC-SOD −/−mice.

Our finding of a modest increase in MAP in conscious and anesthized EC-SOD −/−mice conflicts with the finding of Jung et al. (Jung O et al. 2003 Circ Res 93:622-629) and Jonsson et al (Jonsson L M et al. 2002 Free Radic Res 36:755-758) of similar systemic BPs in EC-SOD +/+ and −/−mice. The difference from previous studies might relate to the use of tail cuff BPs in one study (Jung O et al. 2003 Circ Res 93:622-629) and the use of older mice in the other (Jonsson L M et al. 2002 Free Radic Res 36:755-758). EC-SOD is expressed in blood vessels primarily on the surface of vascular smooth muscle cells and the subendothelial space (Ookawara T et al. 1998 Am J Physiol 275:C840-C847; Faraci F M and Didion S P 2004 Arterioscler Thromb Vasc Biol 24:1367-1373). It contains a heparin-binding domain that binds to proteoglycans expressed on cell surfaces (Chu Y et al. 2005 Circ 112:1047-1053). The finding that SOD activity in Veh-infused EC-SOD −/−mice was undetectable in the plasma and was reduced in the aorta by ˜33% but was unchanged in the kidneys suggests that the increase in oxidative stress observed in EC-SOD −/−mice is primarily because of the loss of EC-SOD in the plasma and partly because of the loss in the aorta, whereas EC-SOD in the kidney makes little, if any, contribution.

Our study has disclosed 2 factors that may contribute to the higher basal levels of MAP in EC-SOD −/−mice. First, the increase in RVR could raise MAP by directly increasing peripheral resistance. The resulting increase in afferent arteriolar resistance could attenuate the ability of pressure/naturesis mechanisms to compensate for the elevation in BP. This possibility is consistent with observations that narrowed afferent arterioles predict the development of hypertension in the spontaneously hypertensive rat (Norrelund H et al. 1994 Hypertens 24:301-308) and accompany hypertension in humans (Tracy R E and Overll E O 1966 Arch Path 82:526-534). Second, oxidative stress could contribute to the higher BPs found in EC-SOD −/−mice, because oxidative stress induces salt sensitivity (Welch W J et al. 2005 Kidney Int 68:179-187; Kopkan L and Majid D S 2005 Hypertens 46:1026-1031), increases vascular reactivity¹⁶ and contributes to Ang II-induced hypertension (Rajagopalan S et al. 1996 J Clin Invest 97:1916-1923; Modlinger P et al. 2006 Hypertens 47:238-244).

Studies of the response of large blood vessels to endothelium-derived relaxation factor/NO demonstrate that EC-SOD exerts major control over vascular O₂.⁻ and bioavailable NO and thereby modulates the endothelium-derived relaxation factor response (Jung O et al. 2003 Circ Res 93:622-629; Chu Y et al. 2005 Circ 112:1047-1053; Cooke C L and Davidge S T 2003 Cardiovasc Res 60:635-642). Because IC-SOD or Cu/Zn-SOD accounts for 60% to 80% of SOD activity in the kidney, we assessed whether it might compensate for a deficiency of EC-SOD. The levels of IC-SOD and Mn-SOD protein were unchanged in the aorta of EC-SOD −/−mice (Jung O et al. 2003 Circ Res 93:622-629), but IC-SOD protein was reduced in the kidney cortex of these mice. During infusion of Ang II, the mRNA and protein for IC-SOD were increased in the kidney cortex of EC-SOD −/−mice which may thereby account for the maintained SOD activity.

An increase in BP, RVR and reactivity of the afferent arteriole to low dose Ang II infusion depends on O₂.⁻, because these effects can be prevented by the SOD mimetic, tempol (Kawada N, et al. 2002 J Am Soc Nephrol 13 :2860-2868; Wang D et al. 2003 J Am Soc Nephrol 14:2783-2789; Welch W J et al. 2005 Am J Physiol 288:H22-H28; Wang D et al. 2004 Circ Res 94:1436-1442). Ang II infusion increases the expression of p22^{phox} and renal NOX-1, and increases NADPH oxidase activity, 8-Isoprostaglandin excretion, and BP. We showed recently that these effects can be prevented by silencing the p22^{phox} gene (Modlinger P et al. 2006 Hypertens 47:238-244). The increase in renal p22^{phox} expression is itself dependent on O₂.⁻, because it can be prevented by coinfusion of tempol (Welch W J et al. 2005 Am J Physiol 288:H22-H28). Apparently, p22^{phox} expression can function as a feed-forward activator of NADPH oxidase during oxidative stress. This concept is consistent with the present finding that the increased expression of p22^{phox} in the kidneys of EC-SOD −/−mice (FIG. 9) is associated with increased markers of oxidative stress (FIG. 7).

Infusion of Ang II into EC-SOD −/−mice failed to increase p22^{phox} expression or NADPH oxidase activity in the kidneys, or the excretion of isoprostanes or MDA but evoked a similar increase in MAP relative to the effects of Ang II in EC-SOD +/+mice (Table 2). The EC-SOD −/−mice had evidence of oxidative stress and a raised MAP before the start of the Ang II infusion. This suggests that an increase in oxidative stress above an elevated level is not required for the increase in MAP during an Ang II slow pressor response in the mouse. In the present study, the paradoxical increase in SOD activity in the kidneys of EC-SOD −/−mice infused with slow pressor doses of Ang II may have protected these mice from an excessive increase in RVR and thereby limited the increase in MAP. In contrast, the reduced SOD activity in their aorta of EC-SOD −/−mice may explain why BP increases more rapidly in these mice during infusion of Ang II at a pressor rate, because aortic EC-SOD plays a major role in vascular mechanisms of hypertension (see FIG. 4 and Jung O et al. 2003 Circ Res 93:622-629).

### Superoxide Anion is Generated Selectively by Endothelin-1 in Resistance Vessels and Enhances their Contractility

Reactive oxygen spice (ROS) and endothelin-1 (ET-1) contribute to angiotensin-induced hypertension. We used isolated mesenteric resistance arteries (MRAs) from EC-SOD knockout (−/−) mice (n=10) and EC-SOD wild type (+/+) mice (n=10), as a model to investigate the hypothesis that microvascular ET-1 and superoxide anion (O₂.⁻) interact to enhance contractility. Tension and O₂.⁻ were quantitated in real time in dihydroethidium (DHE)-loaded MRAs in a myograph equipped with a dual-emission photon detection fluorescence system to measure ethidium (Eth): DHE ratio (E/D) as a direct readout of O₂.⁻ activity. Phenylephrine-induced contractions were similar in MRAs from both groups and did not change E/D ratio. However, ET-1 induced contractions were significantly increased in −/−mice (100±3% vs 66±5%, p<0.01) and were accompanied by a selective increased in E/D ratio only in ECSOD −/−mice (3.7±0.5 vs 0.5±0.8, p<0.01). PEG-SOD normalized augmented ET-1 contraction (64±4% vs 71±3%; p=ns) and prevent an increase in E/D ratio in MRAs from −/− (0.6±0.5 vs 0.5±0.8, p=ns). In summary, ET-1 generates microvascular O₂.⁻ that enhances its vasoconstrictive action. This is normally prevented by vascular metabolism of O₂.⁻ by EC-SOD which therefore emerges as a major microvascular defense against ROS and vasoconstriction and a potential therapeutically target.

### Tempol Corrects Enhanced Contractions and Enhanced Oxidative Stress in the Mouse Model System of SOD Deficiency Caused by Gene Deletion of EC-SOD

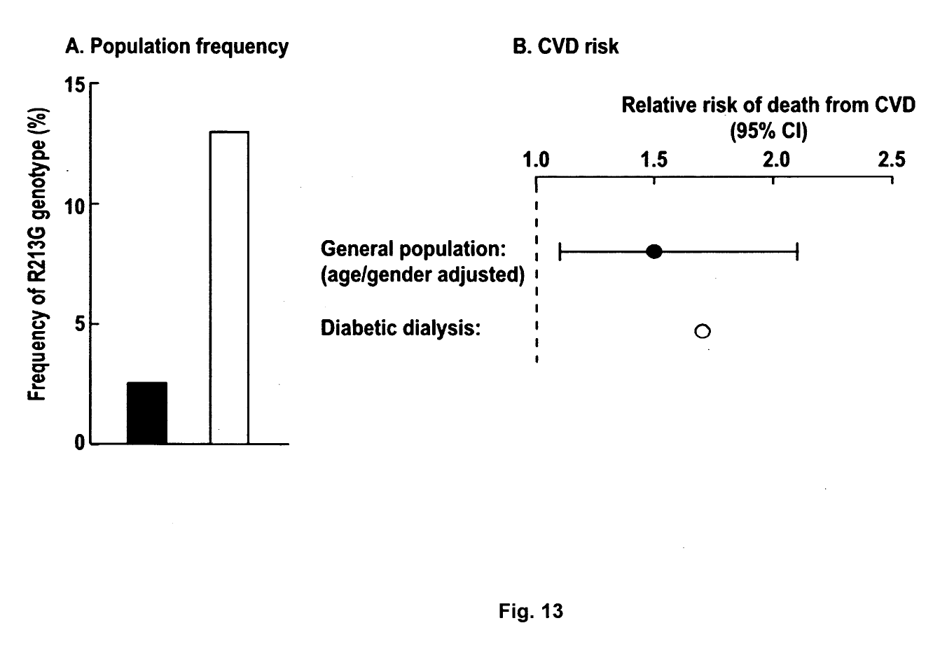

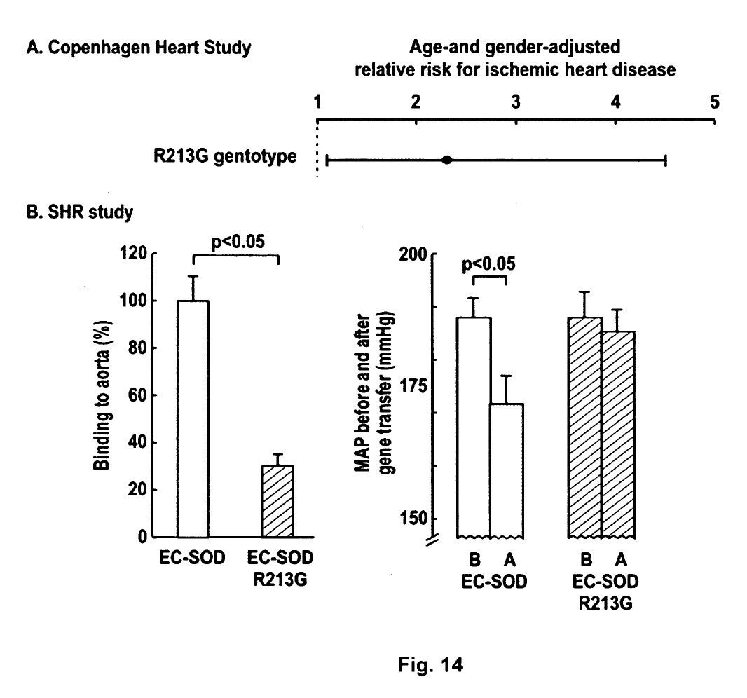

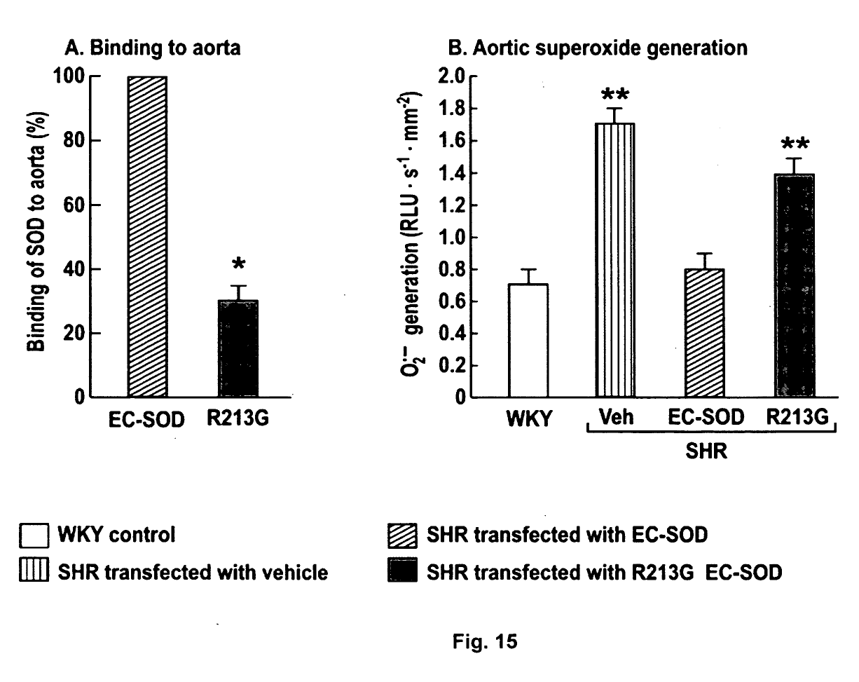

Recent population studies have shown that an R213G single nucleotide polymorphism (SNIP) is present in about 3% of the healthy Danish population (Juul, K. et al. 2004 Cir 109:59-65) and about 13% of a population of diabetic patient on hemodialysis in Japan (Yamada, H. et al. 2000 Nephron 84:218-223). These studies showed an increased relative risk of death from cardiovascular disease of about 50-60% in these populations for those who had the R213G polymorphism (FIG. 13). The Copenhagen Heart Study reported a doubling of risk for subsequent development of ischemic heart disease in subject with this SNIP (Juul, K. et al. 2004 Cir 109:59-65) after controlling for confounding variables. Studies in the spontaneously hypertensive rat (SHR) have shown that rats with the R213G genotype fail to bind EC-SOD to the aorta and that gene transfer of wild-type, but not R213G EC-SOD to SHR reduces the blood pressure (Chu, Y. et al. 2005 Circ 112:1047-1053) (FIG. 14) and the excessive generation of superoxide by the aorta of the SHR (FIG. 15).

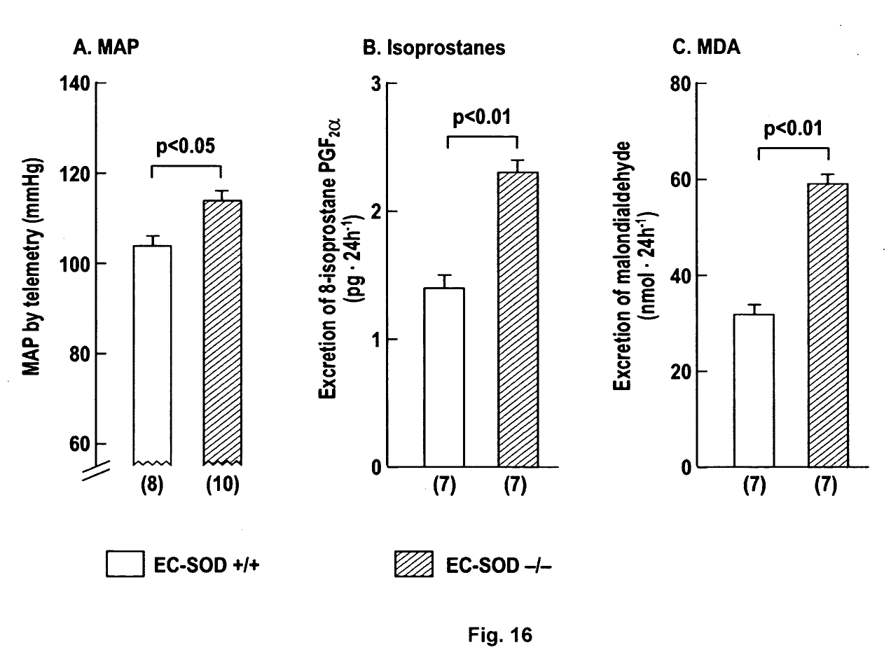

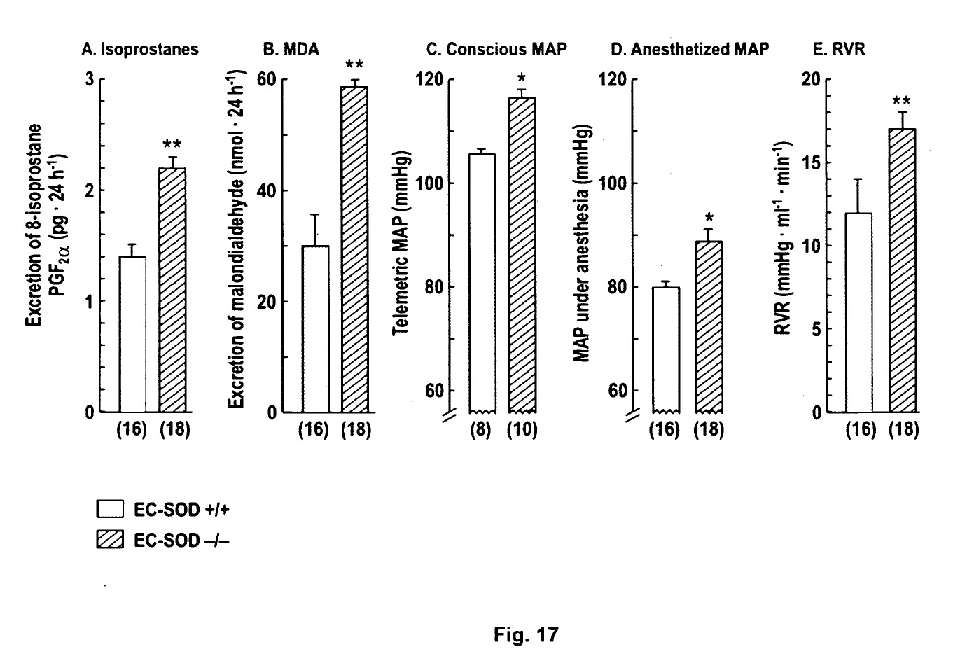

Compared to wild type EC-SOD (+/+), our studies in mice have shown that knockout EC-SOD (−/−) mice have higher levels of mean arterial pressure when measured by telemetry in conscious, unrestrained animals, and increased oxidative stress, as assessed from the steady state excretion of the lipid peroxidation markers, 8-isoprostane PCF_{2α} and malondialdehyde (FIG. 16) (Welch, W. J. et al. 2006 Hypertens 48:934-941). The increase in blood pressure of EC-SOD (−/−) mice was confirmed by direct intra-arterial recordings under anesthetic, and was accompanied by an increase in renal vascular resistance (FIG. 17) (Welch, W. J. et al. 2006 Hypertens 48:934-941).

These studies establish the presence of a SNIP, R213G, in the gene for EC-SOD in the normal population that renders it ineffective by a failure to bind EC-SOD to active sites on the blood vessel wall. The EC-SOD −/−mouse is a model of defective SOD function. It manifests oxidative stress, hypertension and renal vasoconstriction which are precursors of cardiovascular disease (CVD). Finally, human subjects with the inactivating SNIP develop CVD during epidemiologic studies.

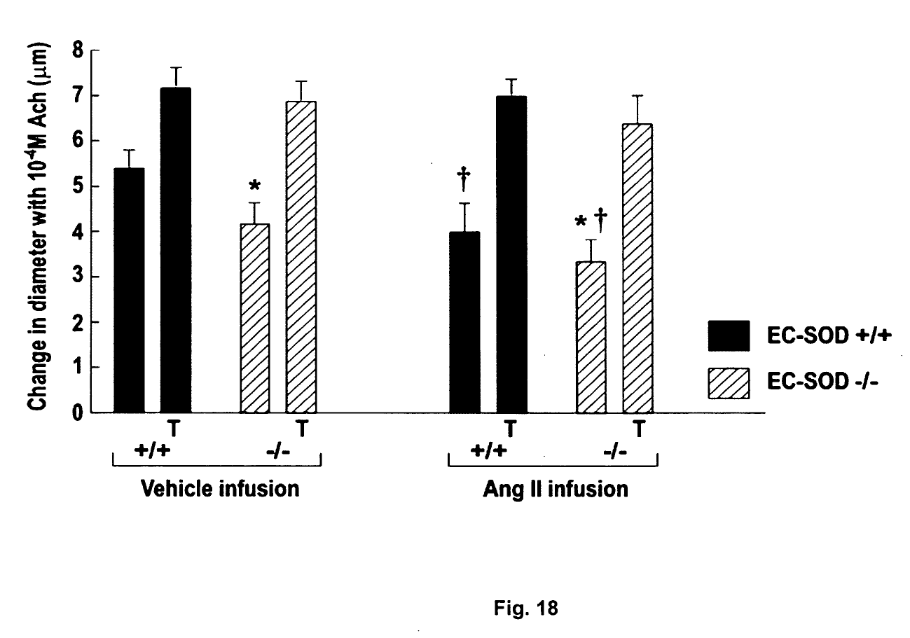

Our current studies have shown that the SOD mimetic drug tempol, when superfused over cremasteric microvessels in the anesthetized mouse, increases that acetylcholine-induced vasodilation response. This effect of acetylcholine is reduced in the EC-SOD (−/−) mouse and in normal and EC-SOD (−/−) mice infused for 12 days with Ang II which causes oxidative stress that bioinactivates nitric oxide which is a mediator of this response. Both the EC-SOD (−/−) phenotype, and mice with prolonged angiotensin-infusion are restored to normal by superfusion with tempol (FIG. 18). This is important since acetylcholine-induced vasodilation is a test of endothelium-dependent relaxation (EDR). Defects in EDR are related to oxidative inactivation of nitric oxide and underlie many CVD risk factors. Thus defects in EDR could be the focus for abnormal vascular responses and increased CVD events in subjects with the R213G SNIP of EC-SOD.

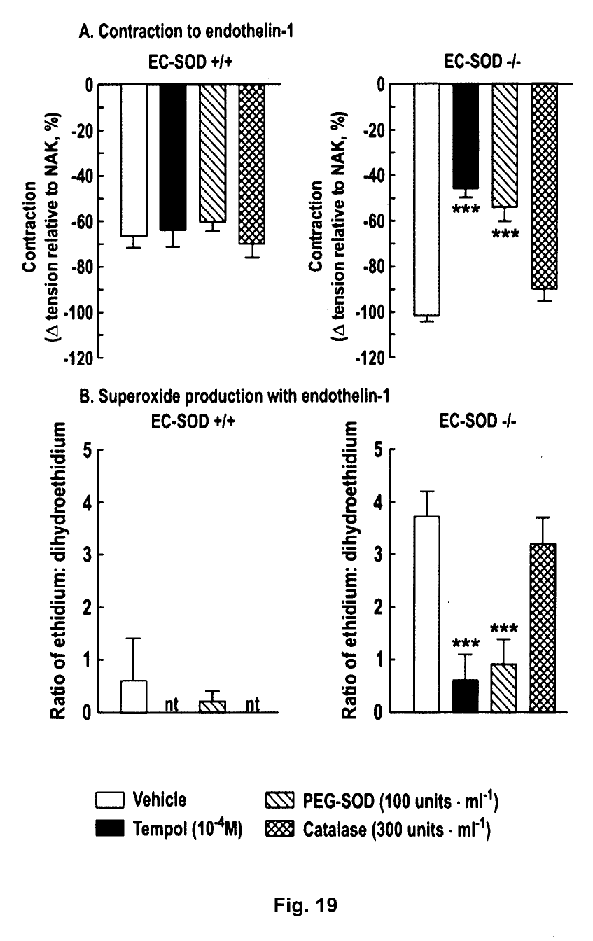

A current study contrasts the responses to endothelin-1 (ET-1) in mesenteric resistance vessels from EC-SOD (−/−) and (+/+) mice. Vessels from EC-SOD (−/−) mice have enhanced contractions to ET-1 that are normalized by bath addition of tempol or PEG-SOD, but not by PEG-catalase (FIG. 19). Parallel studies of vascular superoxide in dihydroethidium-loaded vessels show enhanced conversion to ethidium, indicating enhanced superoxide formation, in vessels from EC-SOD −/−mice. Both the enhanced contraction and the enhanced superoxide are normalized by tempol or PEG-SOD, but not by PEG-catalase. We conclude that in the model system of SOD deficiency caused by gene deletion of EC-SOD in the mouse, tempol can correct enhanced contractions and enhanced oxidative stress. It is thereby envisioned as being effective in preventing hypertension and cardiovascular disease.

### EXAMPLE 1

Animal preparation: The protocol was approved by the Georgetown University Animal Care and Use Committee. Young adult male EC-SOD +/+ and −/−mice were bred from ± founders kindly provided by Dr. Marklund (Umea University, Umea, Sweden). They were developed in a C57B6 mouse background and reproduced and developed normally as described previously (Carlsson L M et al. 1995 Proc Natl Acad Sci USA 92:6264-6268).

For the first series, mice were instrumented with indwelling radiotelemeters (DSI) connected to a cather in a carotid artery and placed within the abdomen 2 weeks before placement of osmotic minipumps as described previously (Kawada N, et al. 2002 J Am Soc Nephrol 13 :2860-2868). Mice were anesthetized with isoflurane (1.0 to 1.5% in 100% O₂) before insertion of telemeters and allowed to recover from the surgery for 12 days before the start of BP recording. Basal recordings of MAP and heart rate (HR) were measured continuously by telemetry for 3 days. Thereafter, mice were anesthetized with isoflurane for insertion of osmotic minipumps (Direct Corp) to deliver Ang II (400 ng⁻¹ kg⁻¹ per minute) or vehicle (Veh) subcutaneously for 2 weeks. An additional group received a higher pressor rate of Ang II infusion (1000 ng/kg⁻¹ min⁻¹, SC). The mean values for 24-hour periods are reported. For subsequent series, mice were prepared without placement of telemeters and Ang II was infused only at the slow pressor rate.

Urine Collection: At day 12 of infusion, mice were placed in metabolic cages (Hatteras Instruments). A 24 hour urine sample was collected in the presence of antibiotics (penicillin G: 0.8 mg; streptomycin: 2.6 mg; and amphotericin B: 5 mg) for excretion of 8-isoprostaglandin F_{2α} and malondialdehyde (MDA), as described (Kawada N, et al. 2002 J Am Soc Nephrol 13 :2860-2868; Schnackenberg C and Wilcox C S 1999 Hypertens 33:424-428).

Renal Function: On day 13 of infusion, mice in series 2 were anesthetized with thiobarbital (Inactin, 50 mg⁻¹ kg⁻¹) and ketamine (40 mg⁻¹ kg⁻¹). Cannulae were placed in the jugular vein (for infusion of fluids and renal function markers), the femoral artery (for direct measurement of MAP and HR), and the bladder (for the collection of urine). A tracheostomy was performed to permit free, uninterrupted respiration of room air. [³H]-Inulin was infused at 0.1 μCi⁻¹ hr⁻¹ for the measurement of glomerular filtration rate. [¹⁴C]-Para-amino hippurate (PAH) was infused at 0.2 μCi⁻¹ hr⁻¹. Blood was collected from the femoral artery and renal vein at the end of the collection period to measure hematocrit and renal PAH extraction. Renal plasma flow (RPF) was calculated from the clearance of PAH factored by its renal extraction. Renal blood flow was calculated from RPF factored by (1-hematocrit). Renal vascular resistance (RVR) was calculated from MAP factored by RBF. In separate groups, plasma was obtained, and the aorta and kidneys were harvested and prepared for further analysis.

Biochemical Assays: NO₂+NO₃ (NOx) was measured in an NO chemiluminescence analyzer (model 270B, Sievers Instruments). 8-Isoprostaglandin F_{2α} (8-Iso) was measured by enzyme-linked immunoassay (Cayman, Inc.) using a method described previously and validated against gas chromatography mass spectrometry (Schnackenberg C and Wilcox C S 1999 Hypertens 33:424-428). MDA was measured from thiobarbituric acid reactive substances (Zepto Metric Inc) as described previously (Chabrashvili T et al. 2003 Am J Physiol 285:R117-R124). NADPH oxidase activity was assessed by measuring O₂.⁻ generation in renal cortex homogenates by lucigenin (5 μmol/L)-enhanced chemiluminescence measured in a luminometer (Porter, Inc., Berthold Autolumat, Berthold Technologies) after the addition of 100 μm NADPH, as described previously (Chen Y et al. 2005 Am J Physiol Renal Physiol 289:F749-F753). Plasma renin activity was measured by radioimmunoassay (Disorin). Plasma renin concentration was measured after the addition of supramaximal concentration (0.5 mg⁻¹ mL⁻¹) of mouse angiotensinogen (Peninsula).

Expression of mRNA and Protein for SOD Isoforms, p22^{phox} and p47^{phox} in Kidney Cortex: The expression of these genes was assessed in homogenates of renal cortex using real-time PCR and Western analysis, as described in detail previously (Chabrashvili T et al. 2003 Am J Physiol 285:R117-R124).

SOD Activity: This activity was evaluated using a modified chemiluminescence technique (Laihia J K et al. 1993 Free Radic Biol Med 14:457-461) from the inhibition of O₂.⁻ signals by mouse plasma, or homogenates of abdominal aorta or kidney cortex after the addition of xanthine (100 μmol/L) and xanthine oxidase (Sigma-Aldrich). The aorta or kidney cortex was dissected in ice cold PBS and homogenized at 3,000 rpm for 30 minutes. The SOD activity of the supernatant was calculated from a standard curve of inhibition of O₂.⁻ generation by Cu/Zn-SOD (Sigma-Aldrich).

Statistics: The differences between EC-SOD +/+ and −/−mice, the effects of Ang II versus Veh infusion, and the interaction (effects of strain on the responses to Ang II) were assessed by 2-way ANOVA. When appropriate, a post hoc Dunnett's t test was applied to detect significant differences between groups. Data are presented as mean±SEM values. Significance is accepted at p<0.05.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A method of treating extracellular-superoxide dismutase (EC-SOD) deficiency in humans which comprises administering tempol or other nitroxide superoxide dismutase (SOD) mimetic to a human in need thereof.

2. A method of treating EC-SOD deficiency in humans comprising
a) identifying a human in need of treatment of EC-SOD deficiency, and
b) administering tempol or other nitroxide SOD mimetic to said human.

3. A method of treating EC-SOD deficiency in humans comprising
a) administering tempol or other nitroxide SOD mimetic to a human, and
b) measuring treatment of EC-SOD deficiency in said human.

4. (canceled)

5. The method of any of claim 1 to 3 wherein said administration is of a nitroxide SOD mimetic other than tempol.

6. The method of any of claim 1 to 3 wherein said administration is of tempol.

7. The method of any of claim 1 to 3 wherein said administration is of 4-amino-2,2,6,6-tetramethylpiperidine-1-oxyl.

8. The method of any of claim 1 to 3 wherein said administration is of 3-carbomoyl-proxyl.

9. The method of any of claim 1 to 3 wherein said EC-SOD deficiency is identified by plasma level of EC-SOD.

10. The method of claim 9 wherein said EC-SOD deficiency is identified by plasma level of EC-SOD of above 200 ng/ml, 250 ng/ml, 300 ng/ml, 350 ng/ml or 400 ng/ml.

11. The method of claim 9 wherein the plasma level of EC-SOD is identified by ELISA assay.

12. The method of claim 9 wherein the plasma level of EC-SOD is identified by superoxide dismutase activity.

13. The method of any of claim 1 to 3 wherein said EC-SOD deficiency is identified by the genetic polymorphism R213G.

14. The method of claim 13 wherein the genetic polymorphism is identified by sequencing, single stranded conformation polymorphism, or mismatch oligonucleotide mutation detection.

15. The method of claim 13 wherein the genetic polymorphism is identified by antibody detection with antibodies to said R213G.

16. The method of any of claim 1 to 3 wherein treatment of EC SOD deficiency is measured by lack of development of a disease related to EC-SOD deficiency.

17. The method of claim 16 wherein the disease is cardiovascular disease.

18. The method of any of claim 1 to 3 wherein the tempol or other nitroxide SOD mimetic is administered orally.

19. The method of any of claim 1 to 3 wherein the tempol or other nitroxide SOD mimetic is administered parenterally.

20. The method of any of claim 1 to 3 wherein the tempol or other nitroxide SOD mimetic is administered dermally.
